(19) United States
(12) Patent Application Publication
(10) Pub. No.: US 2026/0146122 A1
(43) Pub. Date: May 28, 2026

(54) POLYMERS

(71) Applicant: MOLECULAR MEDICAL LTD, Guildford, Surrey (GB)

(72) Inventors: Sean Leo WILLIS, Guilford (GB); Pedro GARCIA, Guilford (GB); Yiqing TANG, Guilford (GB)

(73) Assignee: MOLECULAR MEDICAL LTD, Guildford, Surrey (GB)

(21) Appl. No.: 19/114,556

(22) PCT Filed: Sep. 26, 2023
(86) PCT No.: PCT/GB2023/052494

§ 371 Date: Mar. 24, 2025

(30) Foreign Application Priority Data
Sep. 26, 2022 (GB) 2214014.9
Apr. 21, 2023 (GB) 2305905.8

(51) Int. Cl. C08F 293/00; A61K 9/107; A61K 47/32

(52) U.S. Cl. CPC C08F 293/005; A61K 9/107; A61K 47/32; C08F 2438/01

(57) ABSTRACT

The present invention provides temperature sensitive block co-polymers which may be used inter alia in medical procedures, including therapeutic embolization, compositions comprising the polymers and processes for their preparation. The block copolymers comprise a First Block comprising A monomer and optionally P monomer and a Second Block consisting of N monomer; wherein P monomer is more hydrophobic than A monomer; and wherein the second block is a thermally responsive block.

### INTRODUCTION

The present invention relates to new block co-polymers and to their use particularly in medical applications such as i.a. embolotherapy, drug delivery and other interventions, such as medical and surgical interventions.

Embolic materials are used extensively in vascular interventional radiology. Such materials are typically introduced into blood vessels to block or reduce blood flow to cause tissue necrosis or to prevent loss of blood. They have typically been employed to treat neuro or peripheral vascular diseases, such as aneurysm, arteriovenous malformation and fistula, uterine fibroids, hyper-vascular tumours and to prevent trauma haemorrhage.

In one approach, embolic agents are liquid based materials which polymerise, solidify, precipitate or change phase in the target vessel to form an occlusion. Early versions included solutions of polymers such as ethylene vinylalcohol in a solvent such as DMSO, which dissipates in the bloodstream causing the polymer to precipitate, occluding the vessel. The intravascular delivery of solvents, such as DMSO, is problematic and can cause i.a. endothelial damage, vessel spasm and pain.

Aqueous embolic agents have recently been developed. In one approach these take advantage of the properties of temperature-sensitive polymers, which undergo a phase transition at temperatures above their lower critical solution temperature (LCST) from a soluble hydrated state to an insoluble, dehydrated state. Useful polymers have an LCST of below 37° C., and remain in their soluble state outside the body, whilst, they rapidly become insoluble upon exposure to body tissues at 37° C. This makes delivery of such polymers through fine catheters and needles difficult, because the low cross sectional area of these devices means the polymer is likely to reach its LCST within the tube and hence become a hydrogel, hindering delivery.

NIPAAM-HEMA copolymers have been developed as embolic agents; these polymers are hydrogels above their LCST, but high ratios of NIPAAM to HEMA in these physical hydrogel systems exhibited low frequency strength loss due to viscoelasticity. To increase the gel strength, a high concentration of polymer is used, which in turn often causes catheter blockage due to low LCST and low elasticity. In order to overcome deliverability issues, the polymers are used at a low concentration, however this leads to weak and soft gels, which break up when used in large blood vessels under high shear, may “creep” or move distally and tend to embolise distal to their point of delivery. Moreover, gel fragmentation can lead to off target embolisations, which are dangerous to the patient. Effective proximal embolisation in relatively large blood vessels are therefore challenging to these physical hydrogels. Furthermore, a feature of some of these responsive polymers, is that they tend to exhibit contraction of the polymer after phase change with the concomitant loss of water from the gel, and shrinkage which can contribute to mobility of the embolus.

It would be desirable to develop aqueous liquid embolics that can be formulated easily at low temperatures, around 20° C., that exhibit good gel strength in situ, can be delivered through catheters without blocking them and that rapidly assume a robust gel form in contact with blood or other tissues without fragmentation or shrinkage. It would also be desirable to be able to visualise such polymers readily in the body by a variety of means and to use the gel form for drug delivery if required.

The polymers, compositions and other aspects of the invention described herein address one of more of the issues identified above.

### DESCRIPTION

In a first aspect the invention provides a co-polymer comprising a First Block comprising A monomer and a Second Block consisting of N monomer, wherein the second block is a thermally responsive block. The First Block may comprise both A monomer and P monomer, wherein P monomer is more hydrophobic than A monomer. When both A monomer and P monomer are present the First Block may be an A-P, or P-A block copolymer (i.e. the First Block is an AP or PA di-block co polymer) or it may be a statistical (i.e. mixed) co-polymer of A and P (which is referred to in general terms as A-co-P).

The polymer may comprise a First Block comprising pendant Second Block or it may comprise First Block comprising an extension of Second Block. Where both A and P are present in the First Block as a statistical co-polymer, Second Block is pendant from both A monomer and P monomer. Where A and P are present as a block co polymer Second Block may be pendant from A monomer, from P monomer or from both.

The polymer may be in a linear format or a star format. Linear format polymers either comprise a single First Block or two identical or different First Blocks covalently coupled in a linear reflected arrangement about a central hub. Star polymers comprise m identical or non-identical First Blocks individually covalently coupled to a central hub. Thus for linear polymers m is 1 or 2, for star polymers m is 3 to 10 as described further below.

In a second aspect, the polymer comprises an initiator of polymerisation, the residue of which forms the hub.

Definitions and preferences for A, P, N, x, x′, y, y′, r and q as described below for the second embodiment and elsewhere herein, apply also to this first embodiment.

In a second embodiment the invention provides a block co-polymer of the formula I:

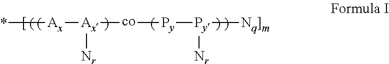

wherein
* represents the site of attachment to the residue of a polymerisation initiator;
A is a hydrophilic or hydrophobic monomer comprising at least one substituent selected from the group consisting of: —OH, —NH, —SH and —COOH;
P is a monomer which is more hydrophobic than A and which optionally comprises at least one substituent selected from the group consisting of: —OH, —NH, —SH, —COOH, alkyl, and aryl groups; N is a monomer forming a thermally responsive block and N_{q} and Nᵣ are terminal, thermally responsive blocks;

The sum of x and x′ is the number of A monomers in a block, and is an integer from 20 to 600, more preferably 30-200, yet more preferably 60 to 120; or 70 to 100.

The sum of y and y′ is the number of P monomers in a block; y and y′ are both 0 when P is absent; when P is present (y+y′) is at least 1, thus (y+y′) is either 0 or is an integer from 1 to 500, preferably (0, 1 or 2) to 100 more preferably 3-30;

The sum of x′ and y′ is the total number of grafts in the AP (First) Block and is either 0 or is an integer from 1 to 300, preferably 6 to 275, more preferably 8 to 130;
q is the number of N monomers in an extension block, and is 0 or is an integer from 1-800, preferably 20-600, more preferably, 50-400;
r is the number of N monomers in a pendant block and is 0, or is an integer from 3 to 500, preferably 5 to 200, and more preferably 5 to 50; and q and r may not both be zero at the same time;
if r>0 then q is an integer not greater than 2*r. preferably no t greater than r.
m is an integer from 1 to 10; preferably from 1 to 6; more preferably from 1 to 4; and yet more preferably is 1, 2 or 3;

Parentheses represent an integral block with hydrophobic/hydrophilic function formed in a sequence of synthesis; and square brackets enclose an arm of branched structures; and wherein the order of A and P may be reversed when A-P is in the form of blocks.

In one preferred arrangement the block co-polymer may have First Block which is an (AP) block comprising both A and P and is an (A-b-P) block, (P-b-A) block or (A-co-P) block and the co-polymer is (AP)-b-N or (AP)-g-N.

In a further preferred arrangement the block co-polymer may have First Block which is an (A) block consisting of only A monomers, and the co-polymer is (A)-b-N or (A)-g-N.

The figures given for x, x′, y, y′ r and q are figures targeted by synthesis. The skilled person will be aware that, when m>1, it is possible to target the synthesis of polymers in which the number of A, P and N monomers present in each arm may not be the same. For example in the case of total target units of A, P and N which are numbers not divisible by the number of arms, m, so that they will be different in each arm. One example of this situation may be if a polymer such as that of the formula:

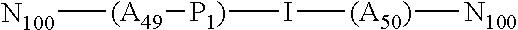

was targeted by synthesis. In this case it is possible to represent the polymer (notionally) as

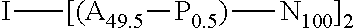

Where, on average, one arm of the polymer has 49.5 A units and 0.5 P units. Ignoring the polymerisation initiator, this can be represented as:

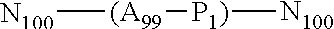

For example, NIPAAM₁₀₀-(HEMA₄₉-HPMA₁)-I-(HEMA₅₀)-NIPAAM₁₀₀ may be targeted by synthesis, and the polymer may be represented (notionally) as

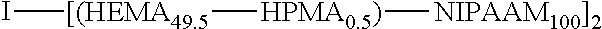

Or, ignoring the polymerisation initiator:

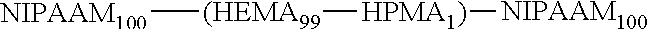

In some embodiments therefore the values of x, x′, y, y′, r and q may be considered to be ranges rather than integers thus:

In some embodiments the sum of x and x′ is in the range 20 to 600, more preferably 30-200, yet more preferably 60 to 120; or 70 to 100.

In some embodiments the sum of y and y′ is in the range 0 to 500, preferably 0, 1 or 2 to 100 more preferably 3-30 or >0 and less than or equal to 10;

In some embodiments the sum of x′ and y′ is in the range (0 or 1) to 300, preferably (5 or 6) to 275, more preferably 8 to 130;

In some embodiments q is in the range 0-800, preferably 20-600, more preferably, 50-400;

In some embodiments r is in the range 0 to 500, preferably 3 to 500, preferably 5 to 200, and more preferably 5 to 50.

In some embodiments r is in the range (0 or 1) to 100, preferably 5 to 80, more preferably 5 to 50.

Bulk polymer compositions may comprise, in addition to a particular target co-polymer, one or more additional polymer products such as other polymers described herein, polymers of differing molecular weight and polymers having alternative block patterns. The target polymer may have x, x′, y, y′ r and/or q values that are integers, but may, as described above, have target polymers in which values for x, x′, y, y′ r and/or q are not the same on each arm. Furthermore synthesis may target a value for x, x′, y, y′ r and/or q within the bulk polymer which may be integers or any fractional value there between. Consequently the skilled person will be aware that the values given for x, x′, y, y′, r and/or q in relation to bulk polymers herein may be considered to be ranges including all fractional values between the limits.

The inventors have identified that, in (A)N polymers, the hydrophobicity of the (A) block can be modulated by incorporating a second, more hydrophobic monomer (P) to increase the hydrophobicity of the block. Increasing the hydrophobicity of the block tends to i.a., improve gel stiffness and reduce gel syneresis, thus polymers having both A and P (ie where (y+y′)>0) are preferred. The A-P block may be present as a block co-polymer having an A-block and a P-block or it may be present as a statistical co-polymer of A and P. Where the A-P block is present as a block co-polymer having an A-block and a P-block, the blocks may be reversed as a (P-A) block.

Where both A and P are present, the N-block may be present as an extension of the A-P block or as a graft, or, in some embodiments as both an extension and a graft. The graft may be pendant from the A monomers, the P monomers or, preferably, both. Where the P monomer is not present, the N-block is present either as an extension of the A-block or as a graft, or, in some embodiments as both an extension and a graft. Preferably the N-block is a graft. Where the P monomer is present, the N-block is present either as an extension of the AP-block (ie the First Block) or as a graft, or, in some embodiments as both an extension and a graft.

Thus in some embodiments (y+y′)>0 and the (AP) block is an (A-b-P) block, (P-b-A) block or (A-co-P) block and the co-polymer is (AP)-b-N or (AP)-g-N.

Since N must always be present, r and q may not be zero at the same time.

The additional P monomers in the AP block make the current physical hydrogel much stronger. The additional hydrophobic block is not thermally sensitive and so catheter delivery becomes easier, even at higher concentration. The formed gel has high storage modulus and can withstand more shear stress from blood flow, therefore, making these systems suitable for proximal delivery and safer to use. Contrary to some earlier polymers it is not necessary to cross link the polymers to modulate their properties.

Whilst the polymer comprises either grafted N blocks or N-blocks present as an extension of the AP block, it may be the case that, during synthesis of graft polymers, a small number of N monomers couple to and extend the AP block. Thus in some cases, where r>0, q may be >0 (rather than 0). Under these circumstances it is expected that q will typically not be greater than 2*r. preferably not greater than r.

The sum of x and x′ is the number of A monomers in a block (A, or AP), and, in some embodiments is an integer from 20 to 600, more preferably 30-200, yet more preferably 60 to 120; or 70 to 100.

The sum of y and y′ is the number of P monomers in a block, and in some embodiments y and y′ are both 0 when P is absent; when P is present (y+y′) is at least 1, thus (y+y′) is either 0 or is an integer from 1 to 500, preferably (0,1 or 2) to 100 more preferably 3-30.

The sum of x′ and y′ is the total number of grafts in the AP (First) Block and is either 0 or is an integer from 1 to 300, preferably 6 to 275, more preferably 8 to 130.
q is the number of N monomers in an extension block, and in some embodiments is 0 or is an integer from 1-800, preferably 20-600, more preferably, 50-400.
r is the number of N monomers in a pendant block and in some embodiments is 0, or is an integer from 3 to 500, preferably 5 to 200, and more preferably 5 to 50; and q and r may not both be zero at the same time;
if r>0 then q is an integer not greater than 2*r. preferably not greater than r.
m is an integer from 1 to 10; preferably from 1 to 6; more preferably from 1 to 4; and yet more preferably is 1, 2 or 3;
x′ is the number of grafts on the A monomer and in some embodiments is 0; or is an integer from 1 to 400, preferably 5 to 200, and more preferably 7 to 100
y′ is the number of grafts of Nᵣ attached to P. In some embodiments, y′ is 0 (when r is 0); or is an integer from 1 to 100, preferably 1 to 75, and more preferably 1 to 30.

In some embodiments a relatively high ratio of A:P is preferred thus in some embodiments, in addition to the above preferences the following proviso applies: A is 70%−(98% or 99% or 99.9%) of the AP block mol/mol, particularly 80−(95% or 99.9%) mol/mol.

When P is present, the ratio (x′+y′)/[(y+y′)+(x+x′)] is the ratio of monomer carrying a graft to total monomer in the (First) block; In some embodiments this ratio is 0.0001 to 1; preferably 0.01 to 1; more preferably 0.1 to 1.

In some embodiments, and optionally in addition to the above preferences the following proviso applies: the ratio of (A) or (AP) to N on a mol/mol basis is 1:0.1 to 1:8; preferably 1:2 to 1:5 and particularly 1:2 to 1:4 In some embodiments either A monomer, or A monomer and P monomer, form a First Block; and N monomer forms a Second Block.

In some embodiments; and optionally in addition to the above preferences the following proviso applies total A (x+x′):P (y+y′):total N is 30-500:(0 or 1)-200:100-600; preferably 30-500: 5-200:100-600 preferably 30-500:5-200:100-600 and more preferably 100-200:10-30:400-500.

In some embodiments where P is not present and where m=1 or 2; A:N is between 0.2:1 and 2:1.

Polymers of the present invention may be linear or they may be star polymers. A linear polymer according to formula I is considered herein to be any polymer in which m is 1 or 2, regardless of the arrangement of the polymer arms on the initiator. The number of arms (m) may be from 1 to 10, preferably from 1 to 6 and more preferably 1, 2 or 3. Thus in particularly preferred arrangements the polymer may be either linear or in a tri arm configuration. Most preferably m is 2 or 3.

The initiators of polymerisation (I) can be any suitable initiator known to those in the art and may be selected for example for its ability to support initiation of the selected number of chains, particularly the initiator is one suitable for atom transfer radical polymerization (ATRP). For example a linear polymer requires the initiator to support initiation from either one or two groups and a star polymer requires the initiator to support initiation from 3 or more groups. Polymers typically retain the residue of the initiator, which in some instances may link the arms of a polymer having 2,3,4,5,6 or more arms. Herein the residue of the initiator is represented by I and the functional group (typically a halide) is lost from the initiator.

Polymer initiators typically comprise either alkyl bromides or alkyl chlorides, where the number of halides is from 1 to 10. Thus for linear polymers, in which m is 1, I may be selected from the group consisting of alkyl α-bromoisobutyrate, benzyl α-bromoisobutyrate, alkyl 2-bromopropionate, 1-phenyl ethylbromide, tosyl chloride, and 2-bromopropanitrile, in which alkyl is C₁-C₁₈ and preferably C₃-C₂. Where m is 2, the initiator may be selected from the group consisting of diethyl meso-2,5-dibromoadipate, ethylene bis(2-bromoisobutyrate), bis[2-(2′-bromoisobutyryloxy)ethyl]disulfide, bis[2-(2-bromoisobutyryloxy)undecyl]disulfide, 2-[2-[2-(2-bromo-2-methylpropanoyl) oxyethoxy]ethoxy]ethyl 2-bromo-2-methylpropanoate, 2-[2-(2-bromo-2-methylpropanoyl) oxyethoxy]ethyl 2-bromo-2-methylpropanoate, and [2-(2-Bromopropanoyloxy)-2-methylpropyl]2-bromopropanoate.

For star polymers where m is 3, initiators may be selected from the group consisting of 1,1,1-tris(2-bromoisobutyryloxymethyl)ethane, glycerol tris(2-bromoisobutyrate), tris(2-bromopropanoic acid)1,2,3-propanetriyl ester, 2,2′,2″-Nitrilotri(ethanol 2-methyl-2-bromopropanoate), and 3,4-bis[(2-bromo-2-methylpropanoyl)oxy]butyl 2-bromo-2-methylpropanoate. Where m is 4, the initiator may be selected from the group consisting of pentaerythritol tetrakis(2-bromoisobutyrate), 3,3,4-tris[(2-bromo-2-methylpropanoyl)oxy]butyl 2-bromo-2-methylpropanoate, and [2,4,5,5-Tetrakis[(2-bromo-2-methylpropanoyl)oxy]-6-methyloxan-3-yl]2-bromo-2-methylpropanoate. Where m is 5 the initiator may be selected from the group consisting of [3,4,5,6-tetrakis[(2-bromo-2-methylpropanoyl)oxy]oxan-2-yl]methyl 2-bromo-2-methylpropanoate and dipentaerythritol hexakis(2-bromoisobutyrate. Where m is 6, the initiator may be, for example 1-O,2-O,3-O,6-O-tetrakis(2-bromo-2-methylpropanoyl)-4-O-[2-O,3-O,4-O,6-O-tetrakis(2-bromo-2-methylpropanoyl)-beta-D-galactopyranosyl]-alpha-D-glucopyranose.

Monomer A may be selected from hydrophilic or hydrophobic monomers comprising at least one substituent selected from the group consisting of: —OH, —NH, —SH and —COOH. In practice however, such substituents may be present in the form of protected functional groups during polymerisation steps or as residues of the functional group when used to couple further groups such as grafts or extension blocks. For example —NH, —SH, and —COOH may be protected during synthesis. Monomer A is preferably selected from the group consisting of acrylates, methacrylates, acrylamides and methacrylamides; more preferably A is selected from the group consisting of an acrylate ester, a methacrylate ester, an N-substituted acrylamide and an N-substituted methacrylamide. In some embodiments, the acrylate esters and the methacrylate esters are independently esters of a C₁ to C₆ alcohol bearing at least one non-esterified hydroxyl group. In some embodiments they may also be esters of a polyethylene glycol having, for example, between 1 and 6 ethylene glycol units, preferably 1 to 3 units. In some embodiments they may be esters of a zwitterionic alcohol (such as 2-methacryloyloxyethyl phosphorylcholine, 2-(N-3-Sulfopropyl-N,N-dimethyl ammonium)ethyl methacrylate, carboxybetaine methacrylate).

In some embodiments the N-substituted acrylamides and N-substituted methacrylamides may be N-substituted by a C₁ to C₆ hydroxyalkane group bearing at least one hydroxyl group. In some embodiments they may be esters bearing non zwitterionic charged groups, such as 2-acrylamido-2-methyl-1-propanesulfonic acid sodium salt (AMPS).

In non-limiting examples, the A monomer may be selected from the group consisting of:
Acrylates: 2-hydroxyethyl acrylate, 3-hydroxypropyl acrylate, 2-hydroxypropyl acrylate and 2-hydroxyisopropyl acrylate.
Methacrylates: 2-hydroxypropyl methacrylate, 1-hydroxy-2-propanyl methacrylate, 2-hydroxyisopropyl methacrylate, 2-hydroxy-2,2-dimethylethyl methacrylate, 1,3-dihydroxypropyl methacrylate (1,3DHPMA), 2,3-dihydroxypropyl methacrylate (2,3DHPMA) [glycerol monomethacrylate (GMA), a mixture of 1,3 and 2,3 DHPMA may be used], dihydroxyethylmethacrylate, hydroxyethylene glycol methacrylate, diethylene glycol mono-methacrylate, 2-hydroxyethyl methacrylate, (1-fluoro-2-hydroxyethyl) 2-methylprop-2-enoate, 3-hydroxypropyl methacrylate, 3-(2-hydroxyethoxy)propyl 2-methylprop-2-enoate, triethylene glycol monomethacrylate, 2,3-butanediol 2-methacrylate, 2-(tert-butylamino)ethyl methacrylate, 2-aminoethyl methacrylate hydrochloride, methacrylic acid 2-[2-[2-[2-(2-hydroxyethoxy)ethoxy]ethoxy]ethoxy]ethyl ester, 2-methacryloyloxyethyl phosphorylcholine, 2-(N-3-Sulfopropyl-N,N-dimethyl ammonium)ethyl methacrylate, carboxybetaine methacrylate and 2-Hydroxy-1-methylethyl methacrylate.
Acrylamides: N-(2-hydroxyethyl) acrylamide, N-(2-hydroxypropyl) acrylamide.
Methacrylamides: N-(2-hydroxypropyl) methacrylamide, N-(2-hydroxyethyl) methacrylamide, hydroxypropyl methacrylamide, N-(1-hydroxybutan-2-yl)-2-methylprop-2-enamide, methacrylic acid dihydroxyethylamide, N-(2-hydroxy-1-methoxyethyl)-2-methylprop-2-enamide, N-(2-hydroxybutyl)-2-methylprop-2-enamide, N-(1-hydroxypropan-2-yl)-2-methylprop-2-enamide, N-(2-aminoethyl) methacrylamide hydrochloride, N-(3-aminopropyl)methacrylamide hydrochloride and 2-acrylamido-2-methyl-1-propanesulfonic acid sodium salt.

The A monomer is preferably selected from the group consisting of 2-hydroxyethyl acrylate, 3-hydroxypropyl acrylate, 2-hydroxypropyl acrylate, 2-hydroxyisopropyl acrylate. 2-hydroxypropyl methacrylate, 2-hydroxy-2,2-dimethylethyl methacrylate, 1,3-dihydroxyproyl methacrylate (1,3DHPMA) 2,3-dihydroxy propylmethacrylate (2,3DHPMA), glycerol monomethacrylate (GMA) dihydroxyethyl methacrylate, hydroxyethylene glycol methacrylate, diethylene glycol mono-methacrylate, 2-hydroxyethyl methacrylate, 3-hydroxypropyl methacrylate, 1-hydroxy-2-propanyl methacrylate, 2-hydroxyisopropyl methacrylate, 3-(2-hydroxyethoxy)propyl 2-methylprop-2-enoate, triethylene glycol monomethacrylate, 2,3-butanediol 2-methacrylate, 2-methacryloyloxyethyl phosphoryl choline, 2-(N-3-Sulfopropyl-N,N-dimethyl ammonium)ethyl methacrylate. N-(2-hydroxyethyl) acrylamide, N-(2-hydroxypropyl) acrylamide, N-(2-hydroxypropyl) methacrylamide, N-(2-hydroxyethyl) methacrylamide, methacrylic acid dihydroxyethylamide, 2-acrylamido-2-methyl-1-propanesulfonic acid sodium salt.

The A monomer is preferably selected from 1,3DHPMA, 2,3DHPMA, GMA, HEMA HEA and HEMAm; particularly 1,3DHPMA, 2,3DHPMA, GMA and HEMA and is particularly HEMA.

Glycerol monomethacrylate (GMA) may be substituted for 2,3HPMA and/or 1,3DHPA. GMA comprises a mixture of 1,3DHPMA and 2,3DHPMA, but 2,3DHPMA is typically around 90% of such preparations.

The P monomer is a monomer that is selected to be more hydrophobic than the A monomer. Hydrophobicity of the monomer is conveniently calculated using the X Log P3 algorithm (Cheng, T.; Zhao, Y.; Li, X.; Lin, F.; Xu, Y.; Zhang, X.; Li, Y.; Wang, R.; Lai, L. “Computation of Octanol-Water Partition Coefficients by Guiding an Additive Model with Knowledge”, J. Chem. Inf Model. 2007, 47, 2140-2148) available at http://www.sioc-ccbg.ac.cn/skins/ccbgwebsite/software/xlogp3/. “More hydrophobic than” in this context means having an X Log P3 value of at least 0.10 higher than the A monomer, preferably at least 0.2 and more preferably at least 0.3 higher.

P is a monomer which is more hydrophobic than A and which optionally comprises at least one substituent selected from the group consisting of: —OH, —NH, —SH, —COOH, alkyl, and aryl groups. In practice however, —OH, —NH, —SH, —COOH groups may be present in the form of protected functional groups during polymerisation steps or as residues of the functional group when used to couple further groups such as grafts. For example —NH, —SH, and —COOH may be protected during synthesis. In some embodiments, monomer P is selected from the group consisting of acrylates, methacrylates, acrylamides and methacrylamides; preferably P is selected from the group consisting of an acrylate ester, a methacrylate ester, an N-substituted acrylamide and an N-substituted methacrylamide.

Non limiting examples of the P monomer include those selected from the group consisting of:
Acrylates: benzyl acrylate, 2-phenylethyl acrylate, 2-hydroxyethyl acrylate, 3-hydroxypropyl acrylate, 2-hydroxypropyl acrylate, 2-hydroxyisopropyl acrylate, 1-phenylethyl acrylate, butyl acrylate, 2-phenylethyl acrylate, methyl acrylate, propyl acrylate, neopentyl acrylate, isooctyl acrylate, benzoxyethylmethacrylate, benzoxyethyl acrylate, 2-methylhexyl acrylate, octadecyl acrylate, and 2-ethylhexyl acrylate.
Methacrylates: 2-methoxyethyl methacrylate, 2-hydroxy-1-methylethyl methacrylate, 2-ethoxyethyl 2-methylprop-2-enoate, isobutyl methacrylate, (3-fluoro-2-hydroxypropyl) 2-methylprop-2-enoate, 2-hydroxy-2,2-dimethylethyl methacrylate, methyl methacrylate, propyl methacrylate, neopentyl methacrylate, 2-acetoxyethyl methacrylate, butyl methacrylate, 2,3-butanediol 2-methacrylate, 3-hydroxypropyl methacrylate, 2-hydroxypropyl methacrylate, 2-hydroxyisopropyl methacrylate, 1-hydroxy-2-propanyl methacrylate, benzyl methacrylate, 2-phenylethyl methacrylate, 1-phenylethyl methacrylate, 3-phenylpropyl methacrylate, phenyl methacrylate, 2-ethylhexyl methacrylate, 4-fluorobenzyl methacrylate, dodecyl methacrylate, fluorobenzyl methacrylate, fluorobenzyl acrylate, (4-methylphenyl)methyl methacrylate, hexyl methacrylate, 2-(4-fluorophenyl)ethyl prop-2-enoate, (2-fluorophenyl)methyl 2-methylprop-2-enoate, and (2-methyl-3-phenylpropyl) prop-2-enoate.
Acrylamides: 2-propenamide,N-(1,1-dimethyl-3-oxobutyl)acrylamide, N-(3-(dimethylamino)propyl) acrylamide, N-(2-(dimethylamino)ethyl) acrylamide, N-[2-(diethylamino)ethyl]acrylamide, N-((dimethylamino)methyl)acrylamide, N-(Hydroxymethyl)acrylamide, N-(isobutoxymethyl) acrylamide, N-(2-hydroxyethyl) acrylamide, N-(2-hydroxypropyl) acrylamide, N-(3-methoxypropyl)acrylamide, N—((S)-1-phenylethyl)acrylamide, N-cyclohexylacrylamide,N-(methoxymethyl)acrylamide, N-(4-chlorophenyl)acrylamide, N-(3-(dimethylamino)-2,2-dimethylpropyl)acrylamide, N-(1,1-dimethylpropynyl)acrylamide, N-(2-fluorenyl) acrylamide, N-(4-methoxyphenyl)acrylamide, N-(4-nitrophenyl)acrylamide, N-(3-nitrophenyl)acrylamide.
Methacrylamides: N-(2-methoxyethyl)methacrylamide, N-(1,1-dimethyl-3-oxobutyl)methacrylamide, N-(3-(dimethylamino)propyl)methacrylamide, N-(2-(dimethylamino)ethyl) methacrylamide, N-[2-(diethylamino)ethyl]methacrylamide, N-((dimethylamino)methyl) methacrylamide, N-(Hydroxymethyl)methacrylamide, N-(isobutoxymethyl)methacrylamide, N-(2-hydroxyethyl) methacrylamide, N-(2-hydroxypropyl) methacrylamide, N-(3-methoxypropyl) methacrylamide, N—((S)-1-phenylethyl)methacrylamide, N-cyclohexylmethacrylamide, N-(methoxymethyl) methacrylamide, N-(4-chlorophenyl)methacrylamide, N-(3-(dimethylamino)-2,2-dimethylpropyl)methacrylamide, N-(1,1-dimethylpropynyl) methacrylamide, N-(2-fluorenyl)meth acrylamide, N-(4-methoxyphenyl)methacrylamide, N-(4-nitrophenyl)Methacrylamide and N-(3-nitrophenyl)methacrylamide.

In a preferred embodiment, the P monomer may be selected from the group consisting of benzyl acrylate, 2-phenylethyl acrylate, 2-hydroxyethyl acrylate, 3-hydroxypropyl acrylate, 2-hydroxypropyl acrylate, 2-hydroxyisopropyl acrylate, 1-phenylethyl acrylate, butyl acrylate, 2-phenylethyl acrylate, methyl acrylate, propyl acrylate, 2-methoxyethyl methacrylate, 2-hydroxy-1-methylethyl methacrylate, 2-ethoxyethyl 2-methylprop-2-enoate, isobutyl methacrylate, methyl methacrylate, propyl methacrylate, neopentyl methacrylate, butyl methacrylate, 2,3-butanediol 2-methacrylate, 3-hydroxypropyl methacrylate, 2-hydroxypropyl methacrylate, 2-hydroxyisopropyl methacrylate, 1-Hydroxy-2-propanyl methacrylate, benzyl methacrylate, 2-phenylethyl methacrylate, 1-phenylethyl methacrylate, 3-phenylpropyl methacrylate, phenyl methacrylate, 2-ethylhexyl methacrylate, 2-propenamide,N-(1,1-dimethyl-3-oxobutyl)acrylamide, N-(3-(dimethylamino)propyl) acrylamide, N-(2-(dimethylamino)ethyl)acrylamide, N-[2-(diethylamino)ethyl]acrylamide, N-((dimethylamino) methyl)acrylamide, N-(Hydroxymethyl)acrylamide, N-(isobutoxymethyl) acrylamide, N-(2-hydroxyethyl) acrylamide, N-(2-hydroxypropyl) acrylamide, N-(3-methoxypropyl) acrylamide, N—((S)-1-phenylethyl)acrylamide, N-cyclohexylacrylamide, N-(methoxymethyl)acrylamide, N-(3-(dimethyl amino)-2,2-dimethylpropyl)acrylamide, N-(1,1-dimethylpropynyl)acrylamide, N-(3-(dimethylamino)-2,2-dimethylpropyl)acrylamide, N-(1,1-dimethylpropynyl)acrylamide, N-(2-methoxyethyl)meth acrylamide, N-(1,1-dimethyl-3-oxobutyl)methacrylamide, N-(3-(dimethylamino)propyl)meth acrylamide, N-(2-(dimethylamino)ethyl)methacrylamide, N-[2-(diethylamino)ethyl]methacrylamide, N-((dimethylamino)methyl)methacrylamide, N-(Hydroxymethyl)methacrylamide, N-(isobutoxy methyl)methacrylamide, N-(2-hydroxyethyl) methacrylamide, N-(2-hydroxypropyl) methacrylamide, N-(3-methoxypropyl) methacrylamide, N—((S)-1-phenylethyl)methacrylamide, N-cyclohexyl methacrylamide and N-(methoxymethyl) methacrylamide.

The P monomer is most preferably selected from 3-hydroxypropyl methacrylate, 2-hydroxyisopropyl methacrylate, 1-hydroxy-2-propanyl methacrylate, or 2-hydroxypropyl methacrylate, or may be a mixture of any two or more of these isomers any of which individually or as a mixture of 2 or more of said isomers is referred to herein as HPMA; N-(2-hydroxypropyl) methacrylamide (HPMAm) or N-(3-hydroxy propyl)acrylamide (HPA) and Butyl methacryate (BMA).

The monomer N is a monomer forming a temperature sensitive block N_{q} or Nᵣ. The blocks preferably have an LCST value in water of 25° C. to 37° C.; preferably 30° C. to 37° C.; and more preferably 30° C. to 35° C.

N block may be selected from the group consisting of poly(N-isopropylacrylamide) (pNIPAAM), poly(N-isopropylmethacrylamide) (pNIPMAM), poly(N,N-diethyl acrylamide), poly(2-(dimethylamino)ethyl methacrylate) (pDMAEMA), poly(PEG methacrylate) (pPEGMA), poly(N-vinylcaprolactam), poly(2-isopropyl-2-oxazoline), poly(vinyl methyl ether), poly(l-lactic acid)-poly(ethylene glycol)-poly(1-lactic acid) (PLLA-PEG-PLLA), hydroxypropylcellulose and poly(ethyleneoxide)-poly(propyleneoxide)-poly (ethyleneoxide) (PEO-PPO-PEO).

N monomer is preferably selected from NIPAAM, NIPMAM, DEA, DMAEMA, N-vinyl caprolactam, 2-isopropyl-2-oxazoline, vinyl methyl ether, 1-lactic acid-poly(ethylene glycol)-poly(1-lactic acid) (PLLA-PEG-PLLA), N-vinylcaprolactam and 2-isopropyl-2-oxazoline, vinyl methyl ether; and is most preferably NIPAAM, NIPMAM, or DEA, particularly NIPAAM or NIPMAM, particularly NIPAAM

In one preferred embodiment A is selected from HEMA, HEA, 1,3DHPMA, 2,3DHPMA, GMA and HEMAm, P is selected from HPMA, BMA, HPAm and HPA and N is selected from NIPMAM, DEA and NIPAAM. In one preferred embodiment A is HEMA, P is HPMA and N is NIPAAM, in another embodiment A is selected from 1,3DHPMA, 2,3DHPMA and GMA; P is BMA and N is NIPAAM, in another embodiment A is selected from 1,3DHPMA, 2,3DHPMA and GMA, P is BMA and N is NIPMAM

The block co-polymer may have First Block which is an (AP) block comprising both A and P and is an (A-b-P) block, (P-b-A) block or (A-co-P) block and the co-polymer is (AP)-b-N or (AP)-g-N.

The linkage between the N blocks and the AP block is typically an ester, amide or ether linkage, depending on the monomers involved. In a preferred embodiment it is an ester linkage.

In one preferred embodiment the block co-polymer comprises a First Block comprising A monomer and optionally P monomer, wherein P monomer is more hydrophobic than A monomer; and a Second Block consisting of N monomer (ie an N homopolymer), wherein the second block is a thermally responsive block; wherein A monomer is selected from the group consisting of acrylates, methacrylates, acrylamides and methacrylamides; P monomer, when present, is selected from the group consisting of acrylates, methacrylates, acrylamides and methacrylamides.

In some embodiments the N block may be selected from pNIPAAM, pNIPMAM, poly(N,N-diethyl acrylamide), pDMAEMA, pPEGMA, poly(N-vinylcaprolactam), poly(2-isopropyl-2-oxazoline), poly(vinyl methyl ether), poly(l-lactic acid)-poly(ethylene glycol)-poly(1-lactic acid) (PLLA-PEG-PLLA), hydroxypropylcellulose and poly(ethyleneoxide)-poly(propyleneoxide)-poly (ethyleneoxide) (PEO-PPO-PEO)

In some embodiments N monomer may be selected from NIPAAM, NIPMAM, DEA, DMAEMA, N-vinyl caprolactam, 2-isopropyl-2-oxazoline, vinyl methyl ether, PLLA-PEG-PLLA, N-vinylcaprolactam and 2-isopropyl-2-oxazoline, vinyl methyl ether; preferably NIPAAM, NIPMAM and DEA, particularly NIPAAM and NIPMAM, thus preferably the N polymer may be pNIPAAM pNIPMAM or pDEA.

Particularly the A monomer is selected from HEMA, HEA, HEMAm, 1,3HHPMA, 2,3DHPMA and GMA; and particularly HEMA, 1,3DHPMA, 2,3DHPMA and GMA; the P monomer is selected from 3-hydroxypropyl methacrylate 2-hydroxyisopropyl methacrylate, 1-hydroxy-2-propanyl methacrylate, or 2-hydroxypropyl methacrylate, or may be a mixture of any two or more of these isomers (HPMA); HPMAm, HPA and BMA; and the N monomer is selected from NIPAAM, NIPMAM and DEA

The polymer may comprise a First Block comprising pendant Second Block or it may comprise First Block comprising an extension of Second Block

In some embodiments the ratio of [First Block](A) or (AP) monomers to [second Block](N) monomers on a mol/mol basis is 1:0.1 to 1:8. In some embodiments the ratio of monomer carrying a graft to total monomer in the (First) block is 0.0001 to 1; in some the ratio of total A:total P:total N is 30-500:(0 or 1)-200:100-600; in some embodiments, in an AP block, A is 70% to 98%, 70%-99% or 70% to 99.9% of the AP block mol/mol.

Particularly the ratio of [First Block](A) or (AP) to [second Block](N) on a mol/mol basis is 1:0.1 to 1:8 and/or in an AP block, A is 70% to 98% or 70% to 99% or 70% to 99.9% of the AP block mol/mol.

In some embodiments the P monomer has an X Log P3 value of at least 0.10 higher than the A monomer.

In any of the above embodiments the polymer may be in a linear format or a star format as described above.

In bulk polymer comprising polymers of this embodiment x, x′, y, y′, r and q may be considered ranges, as described above.

Other features of this preferred embodiment are as described elsewhere herein.

In a further preferred embodiment the polymer is a linear polymer in the form (a): according to formula 2 in which m is 1 or 2, particularly 2.

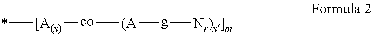

wherein
(x) is 1-800, preferably 50-500, more preferably 60-400;
x′ is 0-400, preferably 1-200, more preferably 5-100;
r is 1 to 500, preferably 5 to 200, and more preferably 10 to 100.

In one embodiment the polymer is a linear polymer in the form (b): according to formula 3 in which m is 1 or 2, particularly 2;

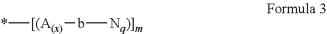

wherein
(x) is 1-800, preferably 50-500, more preferably 60-400; and
q is 1 to 800, preferably 50 to 500

In one embodiment the polymer is a linear polymer in the form (c): according to formula 4, in which m is 1 or 2, preferably 2;

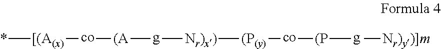

In one embodiment according to formula 4
(x+x′) is preferably 60 to 120; or 70 to 100.;
(y+y′) is 1 to 100 or 2-100; more preferably 1 to 30, 2-30 or 3-30;
(x′+y′) is 1 to 50, preferably 2 to 30, more preferably 5 to 20
r is 1 to 500, preferably 10 to 200.

In a further embodiment of (c) (y+y′) is >0 and <=100, preferably <=50 and more preferably <=30.

In one embodiment the polymer is a linear polymer in the form (d): according to formula 5 and wherein m is 1 or 2, particularly 2.

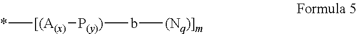

wherein:
(x) is 1-400, preferably 20-200;
(y) is 1-400, preferably 2-100;
q is 1 to 800, preferably 50 to 400; and
m is 2

In a further embodiment of (d) (y) is >0 and <=400, preferably <=100 and more preferably <=50 and particularly les than or equal to 10.

In one embodiment the polymer is a star polymer in the form (e): according to the formula 6; in which m is 3, 4, 5 or 6, particularly 3 or 4 and more particularly 3.

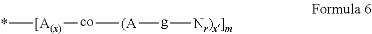

(x+x′) is 1-800, preferably 50-500, more preferably 60-300;
x′ is 1-400, preferably 1-200, more preferably 5-100; and
r is from 1 to 500, preferably 5 to 200, and more preferably 10 to 100.

In one embodiment the polymer is a star polymer in the form (f): according to formula 7, in which m is 3, 4, 5 or 6, particularly 3 or 4 and more particularly 3

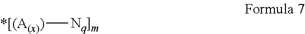

wherein:
(x) is 1-400, preferably 20-200; and
q is 1 to 800, preferably 50 to 500.

In one embodiment the polymer is a star polymer in the form (g): according to formula 8 in which m is 3, 4, 5 or 6, particularly 3 or 4 and more particularly 3.

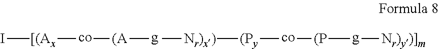

In one embodiment
(x+x′) is preferably 60 to 120; or 70 to 100.;
(y+y′) is (1 or 2) to 100 more preferably 3-30;
(x′+y′) is (1 or 2) to 50, preferably 2 to 30, more preferably 5 to 20
r is 1 to 500, preferably 10 to 200

In a further embodiment of (g) (y+y′) is >0 and <=100, preferably <=50 and more preferably <=30.

In one embodiment the polymer is a star polymer in the form (h): *[(Aₓ-A_{x′}-P_{y}-P_{y′})-N_{q}]m wherein N_{q} is an extension of the (A-P) block; wherein m is 3, 4, 5 or 6, particularly 3 or 4 and more particularly 3; and A and P carry no grafts (r is 0) according to formula 9.

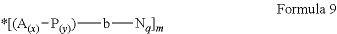

wherein:
(x) is 1-400, preferably 40-200;
(y) is 1-400, preferably (1 or 5)-100
q is 1 to 800, preferably 50 to 500

In a further embodiment of (h) (y) is >0 and <=400, preferably <=100 and more preferably <=50 more preferably less than or equal to 10.

In any of (a) to (f), where both A and P are present the A-P block may be in the form of individual A and P blocks (which may be reversed) or may be in the form of a statistical co-polymer. A and P may be reversed as (P-A).

Forms (c), (d), (g) and (h) are preferred.

In one preferred embodiment A is selected from HEMA, HEA and HEMAm; P is selected from HPMA, HPAm and HPA; and N is selected from DEA and NIPAAM. In one preferred embodiment A is HEMA, P is HPMA and N is NIPAAM.

Particularly preferred polymers include linear polymers of the formula:

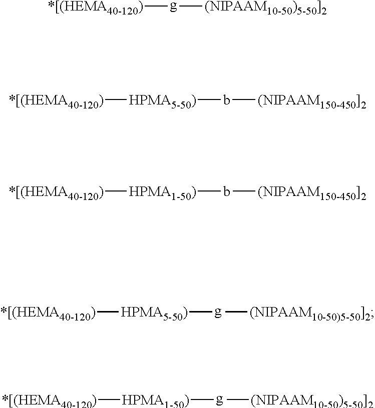

and

Star polymers of the formula:

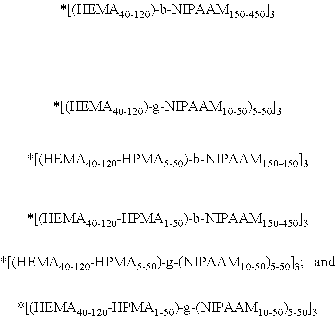

Further particularly preferred polymers include:

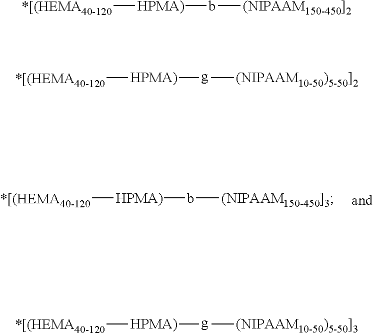

wherein values for the number of HEMA, HPMA and NIPAAM monomers are ranges including all fractional values there between; and wherein the number of HPMA monomers in the AP block is >0 and <=50

Particularly preferred polymers also include:

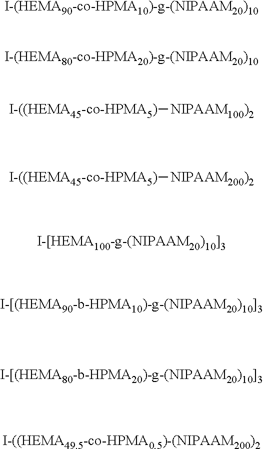

In a further aspect, the invention also provides compositions comprising polymers as described herein. Such compositions include, for example, bulk polymer compositions comprising a polymer described herein. Such bulk compositions retain desirable properties, but may comprise, in addition to a particular target co-polymer, one or more additional polymer products such as other polymers described herein, polymers of differing molecular weight, polymers having alternative block patterns and so on. In some embodiments, such bulk compositions may comprise at least 30%, 40%, 50%, 60% 70%80% or 90% target polymer by weight. Such bulk compositions are a further embodiment of the invention.

Typically such co-polymers or compositions are in a pharmaceutically acceptable form and may for example be sterile and/or pyrogen free.

In one embodiment the composition is an aqueous composition comprising a co-polymer or polymer composition as described herein. In such compositions the polymers of the invention may in sol form (eg soluble or micellar) at a temperature of 25° C. This leads to improved handling since polymers in gel form, such as a hydrogel form, at this temperature are unlikely to be easily deliverable. Such compositions are in the gel form (typically as a hydrogel) at 37° C. The hydrogel is a water swellable but water insoluble polymer. A combination of these features provides a composition that is easy to deliver by a variety of mechanisms, at or close to room temperature (which may be at 20° C., but in practical terms, may fluctuate above or below that from time to time), for example by spraying or through a syringe or catheter, The polymer or bulk polymer, is present in the aqueous compositions in at least 1%, preferably at least 5% more preferably at least 10% w/w. The polymer may be present at up to 30%, up to 50% or up to 90% w/w.

As well as aqueous compositions, the invention also provides dried compositions comprising the co-polymer or polymer compositions herein, by which is meant compositions comprising less than 0.1% water or other solvents w/w, preferably less than 0.01% water or other solvents w/w. This can be achieved by oven drying, spray drying or freeze drying for example.

Such compositions may be compounded with anti-caking agents or dissolution enhancing agents such as glycerol, ethanol, mannitol, glucose, sorbitol, xylitol, trehalose, arabitol, galactitol, fucitol, iditol, inositol, lactose, fructose, sucrose, ribitol, threitol, erythritol, sorbitan, volemitol, isomalt, maltitol, lactitol, citric acid, succinic acid, urea, cholic acid, cholesterol, polysorbate, sorbitan monolaurate, sorbitan monostearate, sorbitan tristearate, decyl glucoside, lauryl glucoside, octyl glucoside, polyvinylpyrrolidone, low molecular weight polyethylene glycol, poloxamer, polyvinyl alcohol, poly(2-acrylamido-2-methylpropane sulfonic acid) or sodium salt, alginate, sodium dodecyl sulfate, sodium lauryl sulfate, ammonium lauryl sulfate, docusate, and triton X-100.

In some cases contrast agents such as iohexol, iopamidol, ioxilan, iopromide, iodixanol, iobitridol, ioversol, diatrizoate, metrizoic acid, iotalamic acid and ioxaglate, may be included in the formulation (or composition). These may also improve solubilisation of the polymer below its LCST in a manner described in more detail below.

Compositions may also comprise a variety of additional components including pharmaceutically acceptable excipients such as small molecules or polymers, including ethanol, glycerol, DMSO, N-methylpyrrolidone, dimethylformamide, diethyl formamide, glucose, lactose, mannitol, hydroxypropylmethylcellulose (HPMC), polyvinylpyrrolidone (PVP), poly(2-Acrylamido-2-methylpropane sulfonic acid) and it salts (eg sodium salt), polyacrylic acid or its salts (such as sodium salt), polymethacrylic acid or its or its salts (such as sodium salt), microcrystalline cellulose, polyvinylpyrrolidone, carboxymethyl starch sodium, croscarmellose sodium, magnesium stearate, polysorbate, poloxamer, sodium lauryl sulfate, hypromellose acetate succinate, alginate, collagen, fibrin, chitosan, gelatin, hyaluronic acid, and cyclodextrin amongst others.

Compositions may comprise at least one imaging agent such as for x-ray fluoroscopy, CT/microCT, magnetic resonance imaging (MRI) or ultrasonic imaging, and/or at least one therapeutic or diagnostic radioisotope

Examples of contrast imaging agents used in X-ray imaging (X-ray fluorography or CT/Micro CT) include metallic particles or powders, such as tantalum, tungsten, rhenium, niobium, molybdenum, gold, and their alloys; barium compounds such as barium sulfate, bismuth compounds such as bismuth subcarbonate, bismuth subsalicylate and bismuth oxychloride. The particles are either in spherical or in irregular shape.

Contrast agents also include radiopaque or iodinated contrast agents, which may be ionic or non-ionic (preferably non-ionic) and include iodinated compounds, such as iohexol, iodixanol, ioversol, iopamidol, ioxilan, iopromide and those mentioned elsewhere herein, or an iodinated oil, such as an ethiodized poppyseed oil (e.g. Lipiodol™).

MRI contrast agents include gadolinium ion containing agents, superparamagnetic iron oxide, ion-platinum particles, and manganese (II) chelates.

Contrast agents used to enhance ultrasound imaging include, but are not limited to, sulphur hexafluoride microbubbles (SonoVue/Lumason™), octafluoropropane (Optison™), perflutren lipid microspheres, CO₂, air, particularly with a lipid/galactose shell, perflexane lipid microspheres (Imagent/Imavist™) and perfluorobutane (Sonazoid™).

Compositions may include at least one therapeutic or diagnostic radio isotope. Imageable or diagnostic radioisotopes include but are not limited to, Ga-67, Ga-68, rubidium-82, molybdenum-99 (Mo-99), thallium-201 chloride, Tc-99, Tc-99m, fluoro-deoxy glucose (FDG) incorporating F-18, In-111, Cu-64, Zr-89, Xe-133, 1-131, Cr-51, Gd-153, and Fe-59. Therapeutic isotopes include but are not limited to Y-90, Ho-166, Lu-167, 1-131, Sr-89 and Sm-153. In some embodiments these can be in the form of chelates.

In some embodiments, chemotherapeutic agents can be incorporated into the compositions. Such agents are selected according to need, but in one approach the compositions may be used in interventional oncology applications, particularly as liquid chemo-embolic agents. Such compositions comprise the polymer and a pharmaceutical active. In this case the possible pharmaceutical actives include the anthracycline class such as doxorubicin, daunarubicin, epirubicin and idarubicin; the camptothecin class such as irinotecan, topotecan, and exatecan; the platins such as cisplatin, oxaliplatin, carboplatin and miriplatin; mitomycin C, nucleoside analogues such as 5-fluorouracil, cytarabine, fludarabine and gemcitabine; multityrosine kinase inhibitors such as sorafenib, sunitinib, regorafenib, brivinb, dasetanib, bosutinib, erlotinib, gefitinib, imatinib and vandetinib; rapamycin; as well as biological actives such as nivolumab (Opdivo), pembrolizumab (Keytruda), ipilimumab, atezolizumab, avelumab, durvalumab, cemiplimab, dostarlimab, relatlimab, spartalizumab, aldesleukin, granulocyte-macrophage colony-stimulating factor (GM-CSF), interferon alfa-2a, interferon alfa-2b (Intron A®), Peginterferon alfa-2b (Sylatron®/PEG-Intron®), imiquimod, olaparib, Poly ICLC (Hiltonol®), pexidartinib; or any combination thereof.

In certain non-limiting examples the co-polymers and compositions of the invention may be used in the treatment of aneurysms, arteriovenous malformation, fistulas, hypervascular tumours, polyps, urinary and faecal incontinence, trauma haemorrhage, tissue separation, tissue bulking, and sealing in the management of wound healing, wound care and wound dressings, for example in burns, and the protection of surface tissues such as damaged skin, from the environment, as dissecting agents, adhesive agents, tissue fillers and cavity fillers (such as for the filling of left atrial appendage).

The compositions are suitable for the embolisation of proximal or distal locations in vascular environments. The invention also relates to the use of the compositions as drug delivery depots.

In some approaches the polymers described herein may be used to provide controlled release formulations of, for example, chemotherapeutics. In one approach this can be achieved by incorporation of charged monomers such as AMPS into the polymer such that the release of the drug is retarded by ionic interactions.

In some embodiments the composition is provided in sterile form. Depending on need this can be achieved by heat or radiation sterilization for example, or may be achieved by reconstitution of a sterile dried composition using sterile aqueous solutions.

A further aspect of the invention provides methods of medical treatment using the polymers described herein.

The polymers and compositions of the invention may be used as embolic agents. A further embodiment therefore provides, a method for the embolisation of a blood vessel in a patient in need thereof, comprising delivering to the lumen of the blood vessel a composition comprising a polymer or polymer composition as described herein. In one approach, the composition is an aqueous composition comprising a polymer (co-polymer or bulk polymer) described herein which may be in the form of a solution or of a micellar suspension and allowing the composition to increase in temperature to a point above its LCST such as to increase the viscosity of the composition and thereby to embolise the blood vessel. In another approach the aqueous composition is in the form of an oily emulsion as described further elsewhere herein or in the form of a suspension of the polymer or polymer composition in an ethiodized oil as described elsewhere herein.

In some embodiments the polymer forms a hydrogel above its LCST. In some embodiments the hydrogel is formed as a string or as a globular deposit.

In some embodiments the method is for the treatment of a hyper-vascular tumor, such as hepatocellular carcinoma (HCC), colorectal cancer or its metastases, neuroblastomas and neuroendocrine tumors; aneurysm, arteriovenous malformation and fistula, uterine fibroids etc.

In some embodiments the method is for the treatment of prostate hyperplasia, by the embolisation of vessels of the prostate. Particularly the prostatic artery or vessels arising therefrom.

In some embodiments the method is for the treatment of osteoarthritis, by the embolisation of vessels of major joints including the knee. Particularly the genicular artery or vessels arising therefrom.

The polymers and compositions of the invention may be used as tissue separation agents. In a further embodiment therefore, the invention provides a method for the separation of a first tissue from a second tissue comprising delivering to a position between the first and second tissues a volume of an aqueous composition comprising a polymer (co-polymer or bulk polymer) described herein in the form of a solution or of a micellar suspension, thereby separating the first tissue from the second tissue, at least in part, and allowing the composition to increase in temperature to a point above its LCST such as to increase the viscosity of the composition and thereby to stabilize the polymer in position.

The polymer and compositions may be delivered using a needle, a catheter or other tubular device. In one approach this technique may be used to separate a first tissue from a second tissue. In one example a first tissue destined for radiation treatment may be separated from a second tissue to be protected from said radiation treatment and thereby to reduce the level of radiation to which the second tissue is exposed. In one particular approach the technique may be used to separate rectal tissue from prostatic tissue destined for radiation treatment and thereby reduce the dose of radiation to which the rectal tissues (such as rectal epithelium) are exposed.

In a further approach polymers and compositions may be used to separate lesion tissue from surrounding tissue to facilitate treatment, for example to provide a submucosal lift of gastrointestinal mucosal lesions (e.g. polyps, adenomas, early-stage cancers) to facilitate surgical excision with a snare or other endoscopic device.

The polymers and composition of the invention may be used as tissue bulking or augmentation agents. In a further embodiment, the invention therefore provides a method for tissue augmentation or bulking, comprising delivering to a position within the tissue a volume of an aqueous composition comprising a polymer described herein in the form of a solution or of a micellar suspension, allowing the composition to increase in temperature to a point above its LCST such as to increase the viscosity of the composition and thereby to stabilize the polymer in position.

The polymer may be delivered using a needle, a catheter or other tubular device. In one approach this technique may be used to bulk tissue from which a polyp subtends. This approach raises the polyp above the tissue making it easier to access for resection. The technique may also be used in the treatment of urinary or faecal incontinence by delivery of a volume of polymer solution to the region of the sphincter muscle or behind the sphincter muscle to improve sphincter closure.

As detailed above the polymers described herein may be combined with contrast agents. In one embodiment such contrast agents include the group of iodinated contrast agents. These are typically polyhydroxylated and poly iodinated compounds. Contrast agents include ionic contrast agents and non-ionic contrast agents. Non-ionic contrast agents are preferred. Contrast agents include iohexol, iopamidol, ioxilan, iopromide, iodixanol, iobitridol, ioversol, diatrizoate, metrizoic acid, iotalamic acid, ioxaglate

The inventors have identified that, rather than forming micelles, temperature sensitive polymers such as those disclosed herein, dissolve in aqueous compositions comprising contrast agents at a temperature below their LCST, such as 25° C. This provides a molecular solution of polymer. Such solutions have a very much reduced turbidity and are substantially clear, do not tend to form hydrogels during delivery and are of lower viscosity than the micellar form at room temperature (20° C.), and consequently easier to deliver. Further, as they are of lower viscosity for a given concentration of polymer, higher concentrations of polymer can be used without blocking catheters, resulting in stronger gels. Such compositions still rapidly become hydrogels at 37° C. Without wishing to be bound by any theory, it believed that the iodinated contrast agents act as a co-solvent to the polymers. This effect may cause the micelles present at lower temperatures (below the LCST) to dissolve or not to form.

Thus a further embodiment of the invention provides a composition comprising (i) a temperature sensitive polymer; preferably those having a LCST between 25 and 37° C.; and (ii) an iodinated contrast agent.

Such compositions may be aqueous compositions or dried compositions. Compositions preferably comprise 1 part polymer to at least 1 part contrast agent by weight preferably at least 2 parts, more preferably at least 5 parts, more preferably at least 10 parts and more preferably still at least 12 parts by weight contrast agent.

Aqueous compositions may be at least 5% w/w polymer more preferably at least 7% w/w, at least 10% w/w, at least 15%, at least 20% or higher. The composition may be up to 30%, up to 50% or up to 80% or up to 90% w/w polymer.

In some embodiments, the ratio of contrast agent to co-polymer or bulk polymer composition is between 0.1 to 10 on a weight for weight basis, preferably 1 to 10, more preferably 1.5 to 5.

At temperatures below the LCST, for example at 25° C., the polymer may be dissolved (or substantially dissolved) in the aqueous composition. The polymer may form a molecular solution in the aqueous composition. The polymer may be in the form of a micellar suspension in the aqueous composition. In some embodiments the aqueous composition may be a clear solution. In some embodiments the solution has substantially no turbidity below the LCST, for example at 25° C.

In a further embodiment the invention provides a method of preparing an aqueous composition comprising a polymer as described herein, the method comprising providing a dried composition comprising the polymer and re-hydrating the polymer in a sterile aqueous medium at a temperature below the LCST of the polymer.

The aqueous medium may be sterile water or saline, for example. The composition may be resuspended below 25° C. or below 10° C. for example. In one embodiment the dried composition comprises an iodinated contrast agent. Reconstitution may comprise preparing a micellar suspension of the polymer, but more preferably the dried composition comprises an iodinated contrast agent and reconstitution comprises dissolving the polymer (or substantially all the polymer) and the contrast agent to prepare a solution of the polymer. In some embodiments reconstitution comprises preparing molecular solution or a clear molecular solution of the polymer.

Iodinated oils, such as an ethiodized poppy seed oil (eg Lipiodol™) are used, in the form of an oily emulsion, comprising an oil phase and an aqueous phase, to provide an embolic composition used in the treatment of (inter alia) hypervascular tumours such as hepatocellular carcinoma. The emulsion may be used without further formulation, but may comprise one or more pharmaceutical actives, particularly anti-cancer agents (eg doxorubicin, irinotecan or platinum drugs) for use in chemoembolization procedures. Although these approaches have been used for many years, there are a number of disadvantages in the use of these emulsions. One particular issue is that the emulsion is only stable for a short period, before beginning to separate into an oil phase and an aqueous phase. This means that the emulsion cannot be prepared in advance and must be prepared in theatre immediately before use. Furthermore, the lack of longer term stability of the emulsion contributes to the rapid dissipation of emulsions in situ so that the embolisation effect is only temporary. In chemo-embolization, dissipation of the emulsion, can also lead to burst release of the active ingredient into the blood stream, which increases off target exposure to the active. The inventors have identified that oily compositions and particularly emulsion compositions comprising polymers and polymer compositions of the invention, such as bulk polymer compositions, have much improved stability, thus a further embodiment of the invention provides a composition comprising a polymer or polymer composition as described herein and an iodinated oil. In particular the iodinated oil is an ethiodised poppy seed oil. Examples of such oils are available under the trade name Lipiodol® (Guerbet, Paris, France) or Vividol™ (Vivere imaging, Hyderabad, India).

In one approach the composition is in the form of an emulsion comprising an aqueous phase and an oil phase wherein the aqueous phase comprises an aqueous composition comprising a co-polymer or bulk polymer as described herein. The composition may also be in the form of the oil and the aqueous phases of the emulsion, in the same container but separated, such as to provide a “ready to prepare” emulsion. In other words the components of the emulsion are present as one liquid volume in which the complete oil and aqueous phases are separate, typically with the aqueous phase floating on top of the oil phase (Lipiodol has a density of 1.28 g/cm at 20° C.).

The ratios of oil to aqueous phase may be chosen to allow preparation of water-in-oil (W/O) or oil-in-water (O/W) emulsions. Oil-in-water-in-oil, or water-in-oil-in water emulsions may also be prepared. In some embodiments, the ratio of aqueous polymer to oil could be 1:1-1:100 v/v, preferably 1:1-1:5 v/v, more preferably 1:1-1:2 v/v to form water-in-oil emulsion.

In some embodiments the ratio of aqueous polymer solution to oil may be 1:0.01-1:1 v/v, preferably 1:0.05-1:0.8 v/v, more preferably 1:0.1-1:0.5 v/v to form oil in water emulsion.

In some embodiments, the polymer concentration in the aqueous solution may be 1%-20%, preferable 3%-15%, more preferably 5%-10%.

The emulsion could be prepared by conventional pumping method through two syringes connected by a 2 or 3-way stopcock or similar connector, or homogenising, or other mechanical mixing devices or methods.

Without wishing to be bound by any theory, it is believed that at ambient temperature, the structured hydrophobic-hydrophilic copolymer chains rearranged at the interface of aqueous-oil emulsions and stabilise the formulation by reducing the surface energy between oil and water. The structure could allow the loading of different therapeutic agents, based on their hydrophobic or hydrophilic properties.

When the emulsion is delivered to the targeted site at body temperature, 37° C., the thermally responsive block is converted to a relatively more hydrophobic form and the original micellar or lamellar structure collapses. The increased hydrophobicity leads to the formation of a more rigid porous gel which functions as an embolisation barriers. Meanwhile the oil contained inside the porous structure provides radiopacity which lasts considerably longer than that seen in traditional emulsions, or may be released as a delivery carrier for hydrophobic drugs, depending on the ratio of aqueous phase to oil and the level of the polymer in the aqueous phase.

Emulsions disclosed herein have an improved stability compared to the same composition lacking the polymer or composition. Stability may be determined by delivering the emulsion composition to a measuring cylinder or a syringe and determining the time taken for half the liquid phase to separate out. This is a simple measure of stability. Alternatively when comparing two compositions of the same volume, it is simpler to determine the time taken for a given volume of aqueous phase to separate out from identical emulsion volumes in identical measuring cylinders. Emulsions described herein are stable for between 10 mins and 2 hrs dependent on the formulation. Emulsions with higher quantities of polymer tend to be stable for longer.

The emulsion formulation may be tuned to have a range of viscosities and thermal response properties by altering the ratio of aqueous solution to Lipiodol. Thus in some embodiments the formulation forms an oily fluid and in others a hydrogel, above the polymer LCST. In some embodiments the formulation is formed as a string or as a globular deposit. The formulation may be used to direct the formulation towards a more distal or more proximal embolisation. In the case of water-in-oil formulations with a high oil volume fraction (High oil fraction to water is above 1:0.9, preferably above 1:0.5), the emulsion tends towards the form of an oily fluid which may be delivered to the distal narrow vasculature. Conversely, oil-in-water formulations with a low oil volume fraction (Low oil fraction to water is below 1:1.1, preferably below 1:1.5) may be more suitable for proximal embolisation of relatively large size blood vessels including those found in vascular diseases such as aneurysms.

The iodinated oil or emulsion formulation may comprise one or more surfactants or emulsion stabilisers. Examples of such additives may include glucose, lactose, mannitol, ribitol, threitol, erythritol, sorbitan, volemitol, isomalt, maltitol, lactitol, cholesterol, polysorbate, sorbitan monolaurate, sorbitan monostearate, sorbitan tristearate, decyl glucoside, lauryl glucoside, octyl glucoside, hydroxypropyl methylcellulose (HPMC), polyvinylpyrrolidone (PVP), polyvinyl alcohol, polyethylene glycol, polyethylene oxide, polyethylene oxide-co-polyproplyeneoxide-co-polyethylenexide (PEO-PPO-PEO), ethylene-vinyl alcohol (EVOH), polyacrylate, polymethacrylate, polyacrylamide, polymethacrylamide, acrylate polymer, polyamide, polysiloxane, polyester, poly urethane, polyvinyl ether, polyvinyl ester, polyglycerol methacrylate, poly(2-acrylamido-2-methylpropane sulfonic acid) or sodium salt, poly(2-acrylamido-2-methylpropane sulfonic acid) and it salts (eg sodium salt), poly(2-methacryloyloxyethyl phosphorylcholine), polyacrylic acid or its salts (such as sodium salt), polymethacrylic acid or its or its salts (such as sodium salt), microcrystalline cellulose, polyvinylpyrrolidone, carboxymethyl starch sodium, croscarmellose sodium, magnesium stearate, polysorbate, poloxamer, sodium lauryl sulfate, hypromellose acetate succinate, alginate, collagen, fibrin, chitosan, gelatin, hyaluronic acid, cyclodextrin, Laponite, sodium dodecyl sulfate, sodium lauryl sulfate, ammonium lauryl sulfate, docusate, and Triton X-100 amongst others.

The emulsion formulations may be comprise compounding agents, imaging agents and/or additional components as described elsewhere herein. In a further embodiment, the emulsion formulations may comprise or may also comprise at least one therapeutic or diagnostic radioisotope and/or at least one chemotherapeutic agent each of which are described elsewhere herein.

Whilst the chemotherapeutic agents appropriate for use with emulsions include those described elsewhere herein, because of the hydrophobic nature of the oil phase, emulsions my also comprise more hydrophobic drugs such taxanes, for example paclitaxel, docetaxel and cabazitaxel.

As well as aqueous compositions, the invention also provides formulations comprising a block co-polymer or bulk polymer as described herein in a dried form, and an ethiodized poppy seed oil, such as Lipiodol® Such compositions may comprise at least 5%, at least 10%, at least 15% or at least 20% w/w of polymer or bulk polymer. This can be achieved by combining the oil with dry polymer powder preferably having a size range between 1 μm and 500 μm preferably between 40 μm and 300 μm.

During the delivery of the formulation into capillary vessels, for example, an embolisation initially occurs as a function of particle size of the polymer, and would subsequently absorb water from blood and/or tissues as the oil liquid phase continues to percolate through the capillary bed thus forming an embolisation at 37° C.

A further aspect of the invention provides processes for the preparation of thermally responsive polymers as disclosed herein and the products of those processes; thus in a first embodiment the invention provides a process for the preparation of a block copolymer comprising polymerizing A monomer and optionally P monomer to provide First Blocks which are either A blocks or AP blocks wherein polymerization is initiated with a polymerization initiator, I, having m sites of polymerization initiation; and reacting N monomers with First Block to provide a block co-polymer comprising First A blocks or AP blocks and Second N blocks; wherein A monomer is reacted at an I:A molar ratio of 1:(20 to 600)m, P monomer is reacted at an I:P molar ratio of 1:(0 to 500)m and N monomer is reacted at an I:N ratio of 1:(1 to 800)m.

A, P and N are as described elsewhere herein.

A monomer may be reacted at an I:A ratio of 1:(20-600)m preferably 1:(30 to 200)m more preferably 1:(60 to 120)m and yet more preferably at 1:(70 to 100)m mol/mol
P monomer may be reacted at an I:P ratio of 1:(0-500)m, preferably 1:(2 to 100)m, more preferably 1:(3-30;)m mol/mol
N monomer may be reacted at a rate of an I:N ratio of 1:(1 to 800)m, preferably 1:(3 to 600)m, more preferably 1:(5 to 400)m mol/mol

In one embodiment, where Second Block may be grafted to First Block and the total number of N monomers grafted to a First Block is 1 to 800; preferably 20 to 600; more preferably 50 to 400.

In some embodiments, where Second Block is present as an extension to First Block and the number of N monomers in the Second Block may be 3 to 500, preferably 5 to 200, and more preferably 5 to 50. AP blocks may be (A-b-P) blocks (P-b-A) blocks or (A-co-P) blocks. The polymer may be AP-b-N or AP-g-N

In some embodiments the individual monomer species are reacted at a molar ratio of A or, where P is present, (A+P) to N of 1:0.1 to 1:8; preferably 1:2 to 1:5 and particularly 1:2 to 1:4.

In some embodiments, where P is present of the First Block monomers reacted, 70% to 99.9% are A monomers; preferably 80-99% or 80-99.9% are A monomers.

In some embodiments N blocks are provided as grafts to the First Block. In some embodiments N blocks are provided as extensions of the First block. In some embodiments N blocks are provided as both grafts and extensions.

Initiators may be selected from those described elsewhere herein.

In any of the above embodiments m may be an integer from 1 to 10, preferably 1 to 6, more preferably 1 to 4 or 1 to 3, particularly preferred embodiments are those in which m is either 2 or 3.

Polymerization may be carried out by RAFT, ATRP or conventional free radical polymerisation, but is preferably carried out by ATRP.

In some embodiments the First Block that reacts with N monomer is in the form of a macro-initiator In some embodiments the process comprises polymerising an A monomer or a combination of A and P monomers to form a first block comprising A and, optionally, P monomers for example of the formula 10

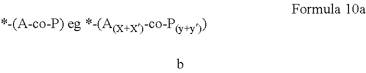

and either
(a) extending an N block, such as an Nq block, from the First Block e.g. an (A_{(x+x′)}-co-P_{(y+y′)}) block to form a polymer of the general formula 11

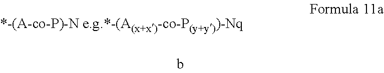

or;
(b) extending at least one N graft, such as an Nᵣ graft from the AP block to form a co-polymer comprising at least one graft of N or Nᵣ according to Formula 11c, e.g. according to formula d;

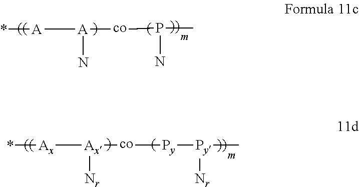

wherein * represents the residue of an optional initiator molecule, configured to support initiation of polymerisation from m functional groups; and wherein m is an integer from 1 to 10.

Polymerisation may be carried out by a variety of approaches including RAFT, ATRP or conventional free radical polymerisation. In a preferred approach the polymerisation is carried out by ATRP.

In one approach to ATRP the process comprises the steps of
(i) Polymerising A monomer or a combination of A and P monomer in the presence of an initiator configured to support ATRP polymerisation from m functional groups to provide a halogenated macro initiator comprising a poly A block or (A-co-P) block comprising the residue of the initiator covalently bound to m copies of either an A polymer or an A-P copolymer, each copy comprising a terminal monomer comprising a Hal group. For example of the formula 12.

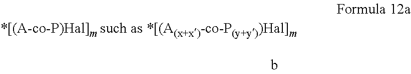

wherein Hal is a halogen selected from Cl and Br, preferably Br and m is the number of polymer arms covalently bound to the initiator; preferably 1 or 2.
(ii) Reacting the halogenated initiator with N monomers in a further ATRP reaction to provide a co-polymer comprising a First Block comprising A monomer and optionally P monomer and a Second Block consisting of N monomer, wherein the second block is an extension of the A block (or AP block).

These are linear polymers as opposed to star polymers.

In a second approach the ATRP process comprises:
(i) Polymerising A monomer or a combination of A and P monomer in the presence of an initiator configured to support ATRP polymerisation from “m” functional groups to provide a star polymer comprising the residue of the initiator covalently bound to m copies of either an A polymer or an A-P copolymer;
(ii) reacting the star polymer with an activated halogen compound, such as α-haloisobutyryl bromide to provide a halogenated macroinitiator in which (in the case of A polymer) halogen is pendant from A monomers; or (in the case of the A-co-P polymer) in which halogen is pendant from both A monomers and P monomers;
(iii) reacting the halogenated macro initiator with N monomer in a further ATRP reaction to provide a co-polymer comprising a First Block comprising A monomer and optionally P monomer and a Second block consisting of N monomer, wherein (in the case of A polymer) second block is pendant from A monomers; or wherein (in the case of the A-co-P polymer) second block is pendant from both A monomers and P monomers.

Typically in an ATRP reaction such as those above, the reaction takes place in the presence of a transition metal halide, typically copper(I) halide. The halogen will typically be the same as that which is present as a functional group on the initiator and the same as that present on the macro initiator. This may be chlorine or bromine but is typically bromine. The reaction typically takes place in the presence of a nitrogen-containing ligand, which binds the transition metal. Typically the reaction takes place in solution from which oxygen has been excluded.

In one example a process for the preparation of a (HEMA-co-HPMA)-block NIPAAM polymer such as (HEMA₍ₓ₎-co-HPMA_{(y)}-block NIPAAM_{q} polymer comprises:
(i) Polymerising a combination of HEMA monomer and HPMA monomer in the presence of a di functional initiator, such as diethyl meso-2,5-dibromoadipate (DMDBA), Cu(I)Br and a nitrogen containing ligand such as 2,2′-bipyridine in a polar solvent such as MeOH to produce a, I-[(HEMA-co-HPMA)Br]₂ macroinitiator, such as a I-[(HEMA₍ₓ₎-co-HPMA_{(y)})Br]₂ macro initiator, in which the terminal monomer of each arm comprises a bromine group (see scheme 1).
(ii) Reacting the brominated macroinitiator with NIPAAM monomers in a further ATRP reaction in the presence of Cu(I)Br in the presence of a nitrogen containing ligand such as 1,4,8,11-tetramethyl-1,4,8,11-tetraazacyclotetradecane (Me4Cyclam) to provide an I-[(HEMA)-co-HPMA)-block NIPAAM]₂ polymer such as an I-[(HEMA₍ₓ₎-co-HPMA_{(y)}-block NIPAAM_{q}]₂ polymer. (See scheme 2)

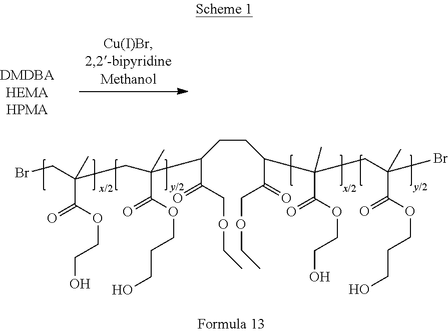

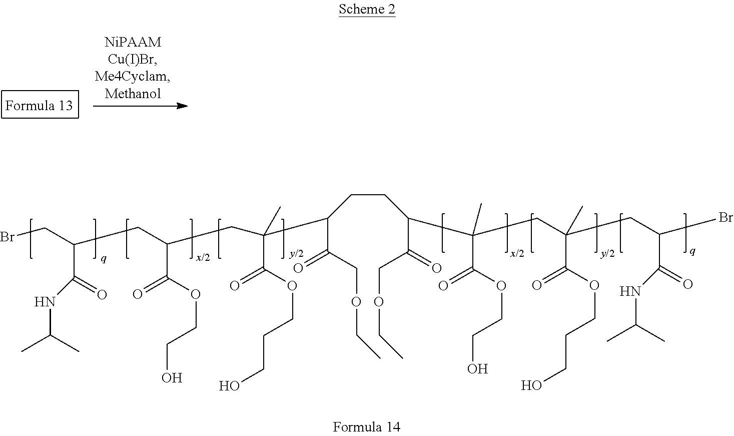

In a second example a process for the preparation of a comb-like tri-arm poly comb-like poly I-[(HEMA-co-HPMA)-graft-(NIPAAM)]₃ polymer such as an I-[(HEMA₍ₓ₎-co-HPMA_{(y)})-graft-(NIPAAMᵣ)_{(x′+y′)}]₃ polymer comprises:
(i) Polymerising a combination of HEMA and HPMA monomers in the presence of a trifunctional initiator such as glycerol tris(2-bromoisobutyrate), Cu(I)Br and a nitrogen containing ligand such as 2,2′-bipyridine to provide a star I-[(HEMA-co-HPMA)]₃ polymer such as a star I-[(HEMA₍ₓ₎-co-HPMA_{(y)})]₃ polymer (formula 15), (Scheme 3).

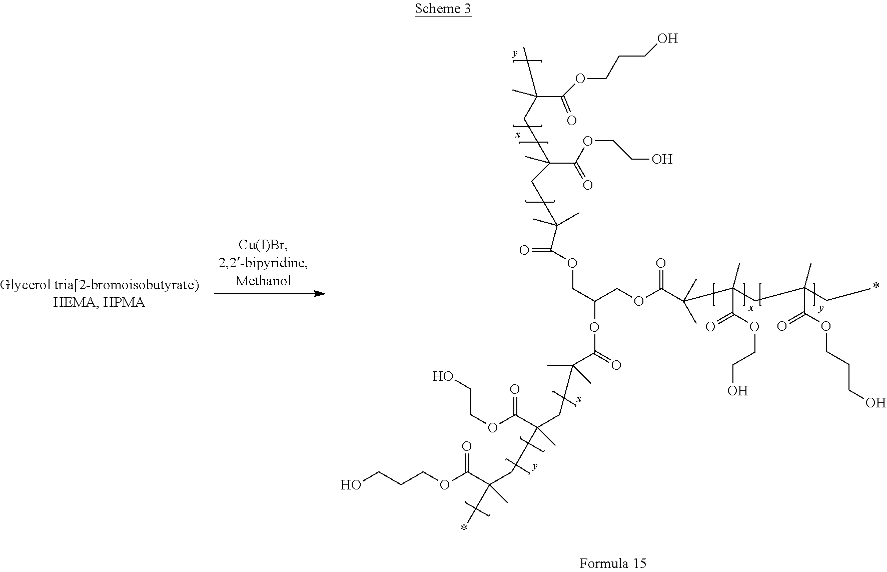

(ii) reacting the star polymer with an activated alkyl bromine compound, such as (x-bromoisobutyryl bromide in the presence of a base, such as triethylamine, to provide a brominated I-[(HEMA-co-HPMA)]₃ macroinitiator, such as an I-[(HEMA₍ₓ₎-co-HPMA_{(y)})]₃ macroinitiator (formula 16), (Scheme 4);

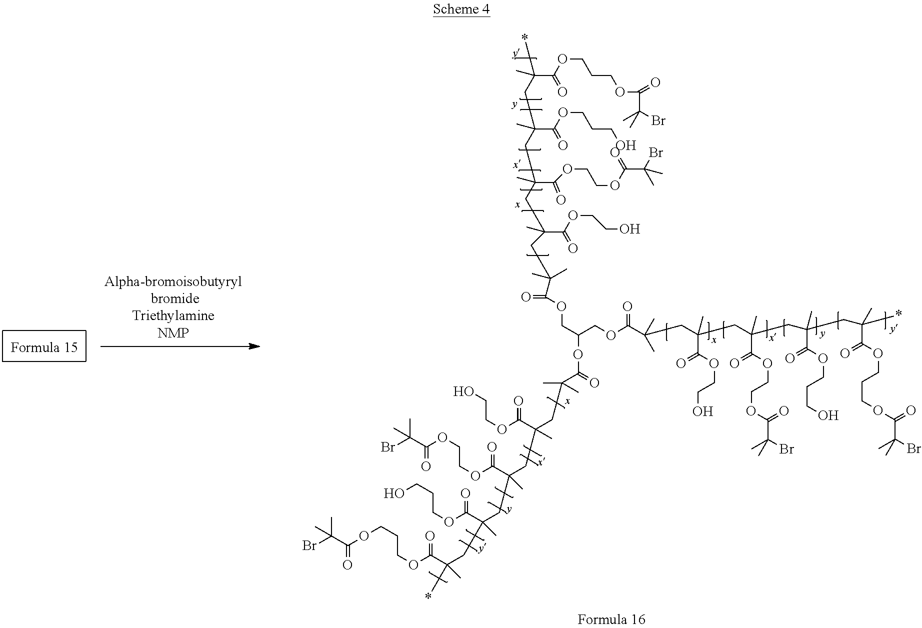

(iii) reacting the brominated I-[(HEMA-co-HPMA)]₃ macroinitiator initiator with N monomer in a further ATRP reaction in the presence of Cu(I)Br and a nitrogen containing ligand such as N,N,N′,N″,N″-pentamethyldiehylenetriamine (PMDETA) in a solvent such as NMP, to provide an I-[(HEMA-co-HPMA)-graft-(NIPAAM)]₃ polymer such as an I-[(HEMA₍ₓ₎-co-HPMA_{(y)})-graft-(NIPAAMᵣ)_{(x′+y′)}]₃ polymer (formula 17), (Scheme 5).

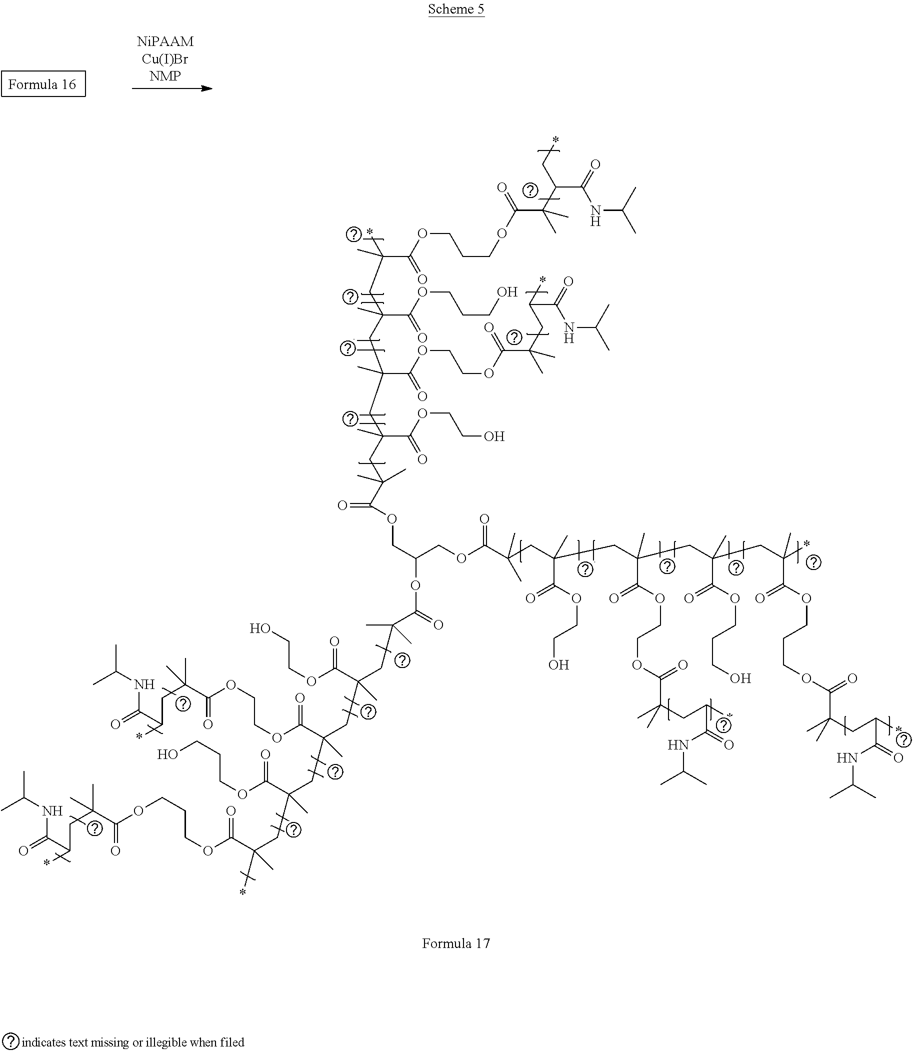

In some embodiments, the process includes the step of drying the resultant polymer so as to provide a dried polymer composition.

In some embodiments the process includes the step of formulating the polymer, by combining it with one or more of the additional components describes elsewhere herein. Formulation may be achieved prior to drying or after drying.

In some embodiments formulation may be achieved by drying any of the compositions described elsewhere herein.

In one embodiment the process may include the step of combining a polymer described herein with one or more contrast agents in solution phase and drying the solution to provide a dried composition of the polymer comprising a contrast agent.

Any of the embodiments or preferred embodiments described above may be used in combination with any other embodiment or preferred embodiment and any of the foregoing may be combined with any aspect of the invention described above.

The invention will now be described with reference to non-limiting experimental examples and figures.

Further embodiments of the invention will be apparent to the skilled person in light of these.

### FIGURES

FIG. 1 illustrates NMR data for synthetic intermediates. FIG. 1a give the NMR spectrum for the Triarm initiator of example 1: glycerol tris(2-bromoisobutyrate). FIG. 1b gives the proton NMR spectrum of the triarm polyHEMA macroinitiator I-[HEMA₁₀₀-Brio]3 of example 3. FIG. 1c gives the proton NMR spectrum of the triarm star comb-like polymer I-[HEMA₁₀₀-g-(NIPAAM₂₀)₁₀]₃ of example 4.

FIG. 2 illustrates aqueous polymer solutions (10% w/w) delivered into PBS at 37° C. (A) is a polymer lacking a P component in the PA block; (B) is a polymer in which P is 10% mole for mole of the A-P block. The polymers are (I-((HEMA₅₀)-b-NIPAAM₂₀₀)₂.) and (I-((HEMA₄₅-co-HPMA₅)-b-NIPAAM-₂₀₀)₂) respectively (see Example 12)

FIG. 3 illustrates Catheter delivery of a liquid sample of I-[HEMA₁₀₀-g-(NIPAAM₂₀)₁₀]₃ in a flow model. Tubing diameter 5 mm, medium temperature 37° C.; initial flow rate 150 mL/min, PBS medium, 2.4 Fr catheter as described in Example 12.

FIG. 4 illustrates delivery of oily emulsions comprising an aqueous solutions of polymer and Lipiodol® into PBS at 37° C. The polymer solution was a 10% solution of I-((HEMA₄₅-co-HPMA₅)-NIPAAM₂₀₀)₂ in water. (A) Ratio of 1:0.5 v/v, (B) Ratio of 1:1 v/v, (C) Ratio of 1:2 v/v (see Example 16). Individual photographs show a time series left to right approximately 30 s apart

FIG. 5 illustrates delivery of a suspension comprising dried polymer particles and Lipiodol® into PBS at 37° C. The polymer was I-((HEMA_{49.5}-co-HPMA_{0.5})-NIPAAM₂₀₀)₂.

FIG. 6 shows a temperature ramping profile of copolymer I-((HEMA_{44.5}-co-HPMA_{0.5})-NIPAAM₂₀₀)₂ aqueous solution (15% w/w) according to Example 13.

### EXAMPLES

In examples, formulae for polymers and intermediates are idealised assuming the reaction runs to completion. In the examples below HPMA is a mixture of isomers hydroxypropyl methacrylates and hydroxyisopropyl methacrylates as outlined further above.

### Example 1: Preparation of Tri-Arm Initiator Glycerol Tris(2-Bromoisobutyrate)

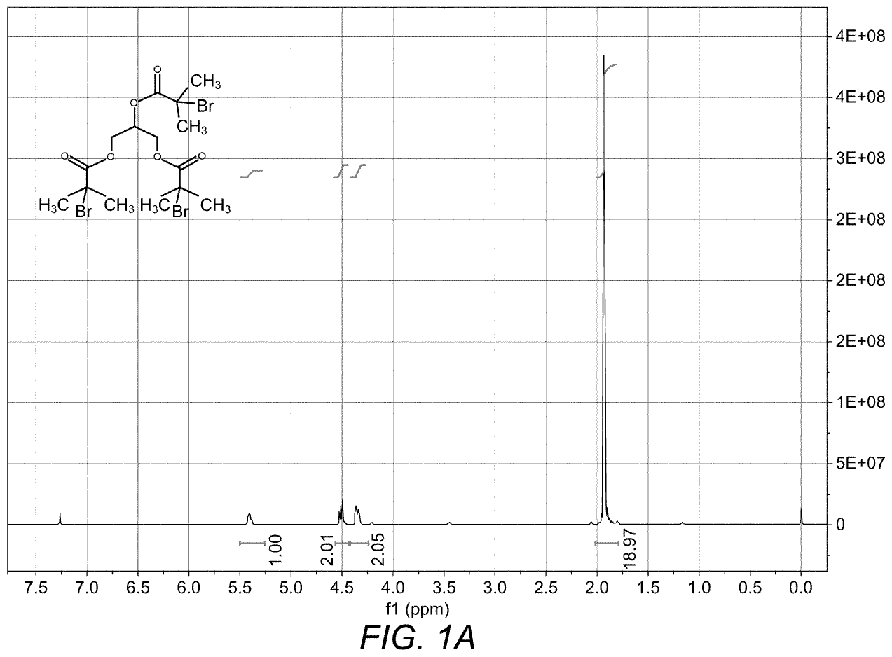

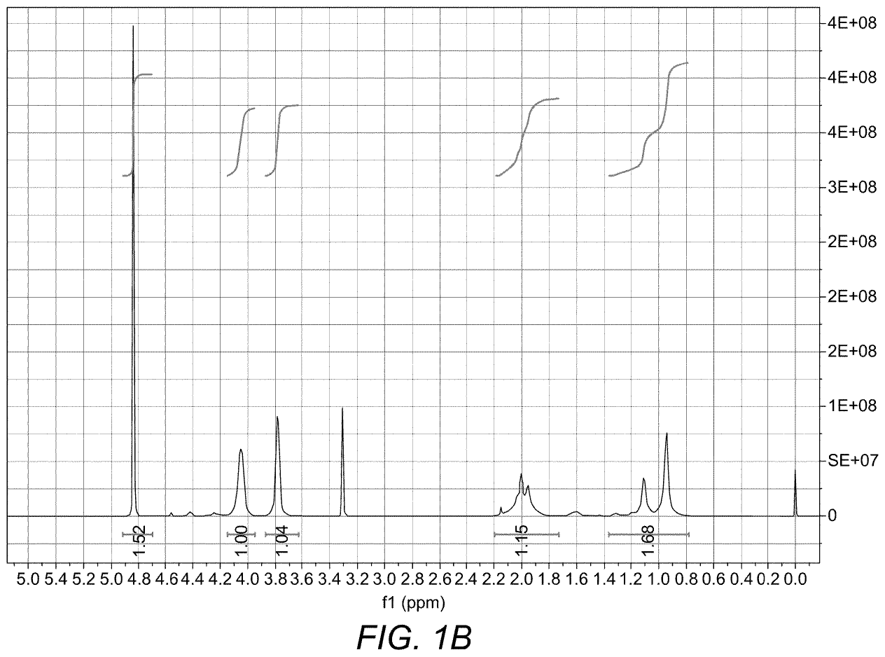

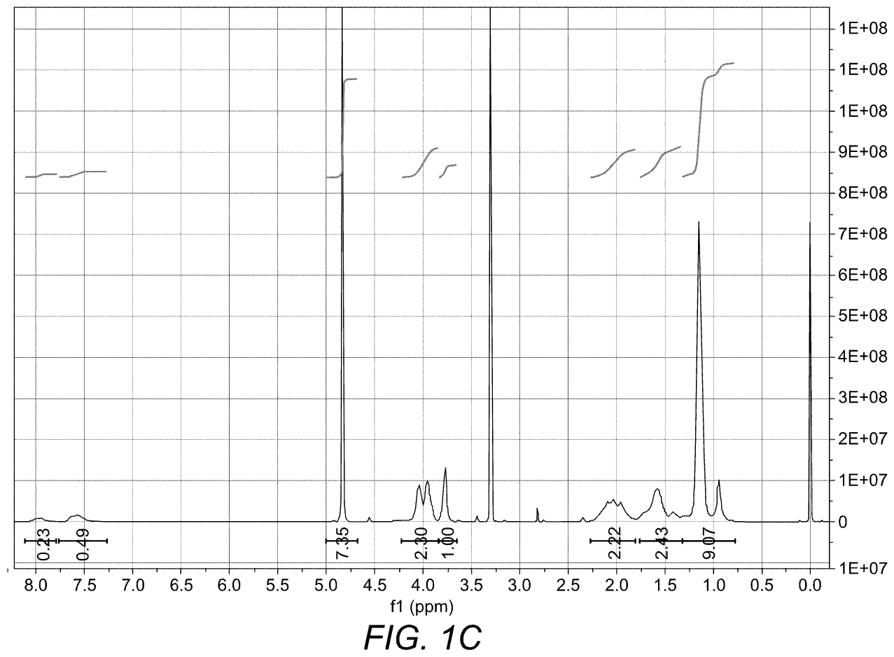

Anhydrous glycerol 5 g (0.054 mol) was charged into a 250 mL round bottom flask, followed by 23 mL of triethylamine (0.168 mol) and 50 mL of anhydrous N-methyl pyrrolidinone. The flask was placed in an ice-water bath and stirred with a magnetic stirrer for 20 min. Then 38 g (0.165 mol) of α-bromoisobutyryl bromide was added dropwise through a dropping funnel over a period of about 30 min. The reaction was stirred at room temperature overnight. The reaction mixture was then filtered to remove triethylamine salt, then the solution was thoroughly mixed with saturated aqueous NaCl solution. Ethyl acetate was used to extract the water solution three times, and the combined organic phase was dried with MgSO₄ overnight. The ethyl acetate solution was removed with a rotary evaporator, and the product vacuum dried for 24 hr to provide a pale yellow wax like solid. The ¹H NMR spectrum for this product is shown in FIG. 1).

### Example 2: Synthesis of Tri-Arm Poly HEMA Homopolymer by ATRP

A typical synthesis procedure of tri-arm poly(I-(HEMA₁₀₀)₃) is given below. 0.2 g (0.37 mmol) of initiator prepared in Example 1, was mixed with 2-hydroxyethyl methacrylate 14.5 g (111 mmol) and 15 mL of methanol in a 100 mL of three-neck round bottom flask. After the mixture was degassed by nitrogen gas under magnetic stirring for 60 min, 0.16 g (1.11 mmol) of CuBr and 0.35 g (2.22 mmol) of 2, 2′-bipyridine were added into the flask to start polymerisation under nitrogen atmosphere. It is observed that the temperature of the reaction mixture increased in the first hour accompanied by a gradual increase in solution viscosity. After about 20 hr, the reaction was stopped by exposure to the air and diluted with methanol. The mixture was passed through a silica gel column to remove the copper catalyst, and the methanol was removed by rotary evaporation followed by vacuum drying overnight at 40° C. to receive 10.6 g solid.

### Example 3: Synthesis of Tri-Arm Poly HEMA Homopolymer Macroinitiator

In a 250 mL round bottom flask, 15 g of tri-arm poly(I-(HEMA₁₀₀)₃) obtained from the reaction of Example 2 was dissolved into 50 mL of anhydrous N-methyl pyrrolidinone under magnetic stirring. Then 1.74 mL of triethylamine was added. The flask was placed in an ice-water bath, then 1.41 mL of α-bromoisobutyryl bromide to target 10 unit of bromide per arm was added dropwise through a syringe over about 10 min. The reaction solution became turbid quickly and was stirred at room temperature overnight. The reaction mixture was precipitated dropwise into deionised water 500 mL to remove NMP and triethylamine salt. The collected solid was dissolved in 30 mL of NMP and the precipitation was repeated. The solid was collected by dissolving in MeOH followed by rotary evaporation and vacuum drying at 40° C. overnight. 12.0 g of polymer was produced (Formula 18).

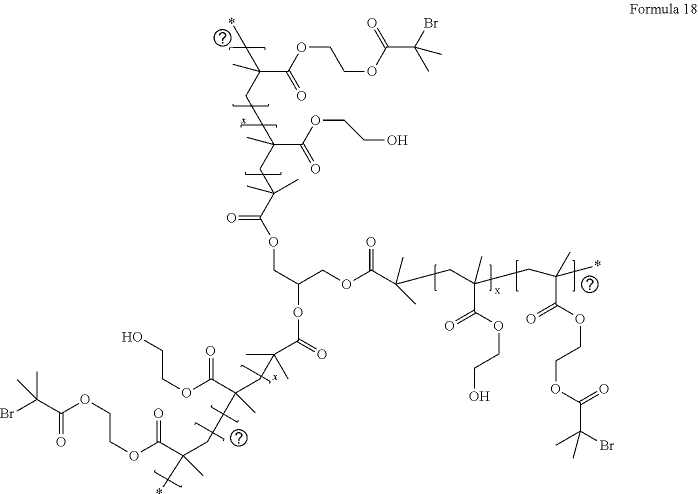

### Example 4: Synthesis of Comb-Like Tri-Arm Poly(HEMA-g-NIPAAM) Copolymer by ATRP

A series of tri-arm poly(I-(HEMA-g-NIPAAM)₃) copolymers were synthesised having varying levels of HEMA and NIPAAM polymerisation, using ATRP. A typical procedure is given below.

In a 100 mL three-neck round bottom flask, 3.0 g of macroinitiator of polyHEMA prepared in Example 3 was dissolved in a mixture of 40 mL of NMP and 30 mL of deionised water. 4.68 g of NIPAAM monomer and 0.36 g of catalyst N,N,N′,N″,N″-pentamethyldiehylenetriamine (PMDETA) were then added. The solution was degassed with nitrogen for 1 hr, and the flask was placed in ice-water bath to allow the mixture temperature to reach 5° C. CuBr catalyst was added under nitrogen flow to start the polymerisation. The solution viscosity increased with time. After about 18 hr, the reaction was stopped by exposure to the air and diluted with 20 mL of NMP. The mixture was dialysed in a dialysis bag against water for four days with water change every 12 hr. The translucent solution obtained was freeze dried, and 6.9 g of white polymer solid (Formula 19) was received at the end. The proton NMR spectrum of this product is given in FIG. 1c.

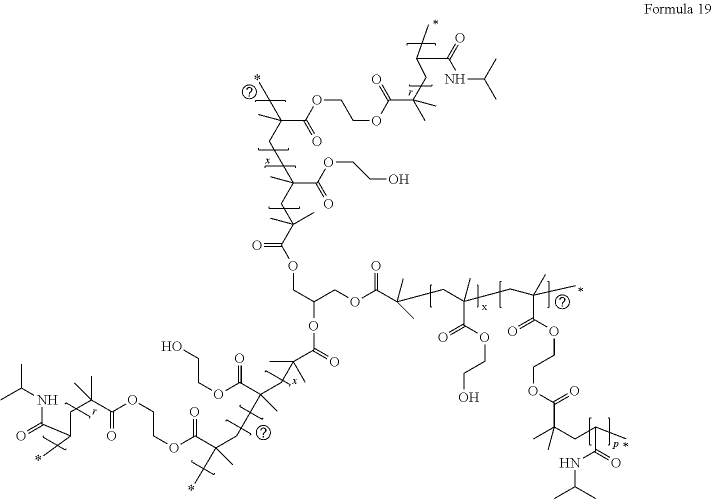

### Example 5: Synthesis of Tri-Arm Poly(HEMA-NIPAAM) Copolymer by Chain Extension Using ATRP

A typical synthesis procedure of tri-arm poly(I-(HEMA₁₀₀-NIPAAM₁₀₀)₃) is given below.

In a 100 mL three-neck round bottom flask, 2.1 g of macroinitiator polyHEMA prepared in Example 2 and 1.81 g of NIPAAM monomer were dissolved in 5 mL of NMP. The mixture was degassed with nitrogen for 60 min and CuBr catalyst (0.023 g) and 1,4,8,11-tetramethyl-1,4,8,11-tetraazacyclotetradecane (Me4Cyclam) 0.041 g were added and polymerisation was allowed to proceed overnight. The reaction mixture was then dialysed against deionised water for four days to remove unreacted monomer and catalyst. The solution was freeze dried and 2.4 g of polymer received.

### Example 6: Synthesis of Linear Copolymer I-((HEMA₄₅-co-HPMA₅)-NIPAAM₁₀₀)₂

(a) Preparation of poly I-(HEMA₄₅-co-HPMA₅)₂ copolymer.

In a 100 mL three-neck round bottom flask, 0.3 g (0.83 mmol) of the initiator, diethyl meso-2,5-dibromoadipate, was mixed with 2-hydroxyethyl methacrylate 9.76 g (75 mmol), hydroxypropyl methacrylate 1.20 g (8.3 mmol) and 12 mL of methanol. HPMA was supplied by Merck (Catalogue No. 268542) and is a mixture of isomers hydroxypropyl methacrylate and hydroxyisopropyl methacrylate. After the mixture was degassed with nitrogen gas under magnetic stirring for 60 min, 0.24 g (1.67 mmol) of CuBr and 0.52 g (3.33 mmol) of 2,2′-bipyridine were added into the flask to start polymerisation under nitrogen atmosphere. It was observed that the temperature of the reaction increased slightly with time and the solution viscosity increased gradually until the magnetic stirring bar stopped moving. After about 18 hr, the reaction was stopped by exposure to the air and diluted with methanol. The mixture was passed through a silica gel column to remove the copper catalyst, and the methanol was removed by rotary evaporation followed by vacuum drying overnight at 40° C. to receive dry polymer solid.

(b) Preparation of I-((HEMA₄₅-co-HPMA₅)-NIPAAM₁₀₀)₂

In a 100 mL three-neck round bottom flask, 3.0 g of macroinitiator poly(HEMA-co-HPMA) prepared above and 5.0 g of NIPAAM monomer were dissolved in 25 mL of MeOH. The mixture was degassed with nitrogen for 1 hr, then CuBr catalyst 0.06 g and Me4Cyclam 0.114 g were added to start the polymerisation. The reaction was left running overnight. The mixture was then dialysed against deionised water for four days to remove unreacted monomer and catalyst. The solution was freeze dried and the linear polymer solid was recovered. The polymer may also be written NIPAAM₁₀₀-(HEMA₉₀-co-HPMA₁₀)-NIPAAM₁₀₀.

### Example 7. Synthesis of Linear Copolymer poly(NIPAAM₂₀₀-HEMA₉₉-HPMA₁-NIPAAM₂₀₀)

A typical synthesis procedure of linear poly(HEMA₉₉-st-HPMA₁) is given below. 1.5 g (4.17 mmol) of initiator diethyl meso-2,5-dibromoadipate was mixed with 2-hydroxyethyl methacrylate 53.7 g (412.81 mmol) and hydroxypropyl methacrylate 0.6 g (4.17 mmol) in a 250 mL of three-neck round bottom flask, followed by adding 55 mL of methanol. After the mixture was degassed by nitrogen gas under magnetic stirring for 60-120 min, 1.20 g (8.34 mmol) of Cu(I)Br and 2.60 g (16.68 mmol) of 2, 2′-bipyridine were added into the flask to start polymerisation under nitrogen atmosphere. It is observed that the temperature of the reaction mixture increased in the first hour accompanied by a gradual increase in solution viscosity. After about 20 hr, the reaction was stopped by exposure to the air and diluted with methanol. The mixture was passed through a silica gel column to remove the copper catalyst, and the methanol was removed by rotary evaporation. After further vacuum drying overnight at 40° C., 45 g solid was received.

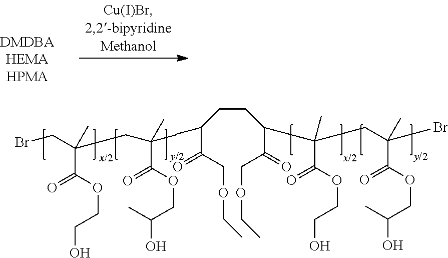

To prepare target poly(NIPAAM₂₀₀-HEMA₉₉-HPMA₁-NIPAAM₂₀₀) copolymer, in a 250 mL three-neck round bottom flask, 15 g of poly(HEMA₉₉-st-HPMA₁) macroinitiator obtained from the reaction above was dissolved into 72 mL of methanol under magnetic stirring. Then 50.7 g of N-isopropylacrylamide monomer was added into the flask and dissolved. After the mixture was degassed with nitrogen for 60-120 min, the flask was placed in an ice-water bath, and 0.32 g (2.24 mmol) of Cu(I)Br and 0.58 g (2.24 mmol) of 1,4,8,11-Tetramethyl-1,4,8,11-tetraazacyclotetradecane were added to start polymerisation. The reaction solution became viscous quickly and was stirred overnight under nitrogen atmosphere. After about 20 hr the reaction was stopped by exposure to air and the addition of 100 mL of methanol to dilute the viscous solution. The polymer was purified by ultrafiltration against water and the obtained solution was freeze-dried over 48 hr. At the end about 54 g of solid was collected. This polymer may also be referred to as I-((HEMA_{45.5}-HPMA_{0.5})-NIPAAM₂₀₀)₂

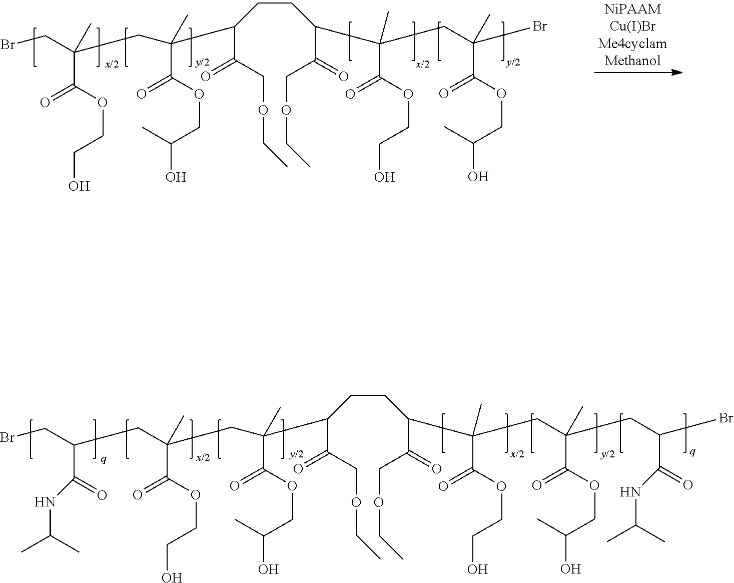

### Example 8: Synthesis of Comb-Like Copolymer Poly(HEMA-HPMA-Graft-NIPAAM)

### (a) Synthesis of Poly(HEMA-HPMA) Macroinitiator

In a 250 mL round bottom flask, 8 g of poly I-(HEMA₄₅-HPMA₅)₂ obtained from Example 6a was dissolved into 50 mL of anhydrous N-methyl pyrrolidinone (NMP) under magnetic stirring, followed by adding 2.72 mL of triethylamine. The flask was placed in an ice-water bath, then 2.20 mL of α-bromoisobutyryl bromide was added dropwise over about 20 min to target 10 units of bromide per chain. The reaction solution became turbid quickly and was stirred at room temperature overnight. The reaction mixture was precipitated dropwise into deionised 500 mLwater to remove NMP and triethylamine salt. The collected solid was dissolved in 30 mL of NMP and the precipitation was repeated. The solid was collected and vacuum dried at 40° C. for two days.

(b) Synthesis of comb-like copolymer I-[(HEMA₄₅-HPMA₅)-g-(NIPAAM₂₀)₅]₂

In a 100 mL of three-neck round bottom flask, 3.0 g of macroinitiator poly(HEMA-HPMA) prepared above and 5.0 g of NIPAAM monomer were dissolved in 15 mL of NMP. The mixture was degassed with nitrogen for 60 min, then CuBr catalyst 0.32 g and PMDETA 0.57 g were added to start the polymerisation. The reaction was left running overnight and then dialysed against deionised water for four days to remove unreacted monomer and catalysts. The solution was freeze dried and polymer solid was recovered. The formula can also be expressed as poly(HEMA₉₀-HPMA₁₀-g-(NIPAAM₂₀)₁₀) (FIG. 20)

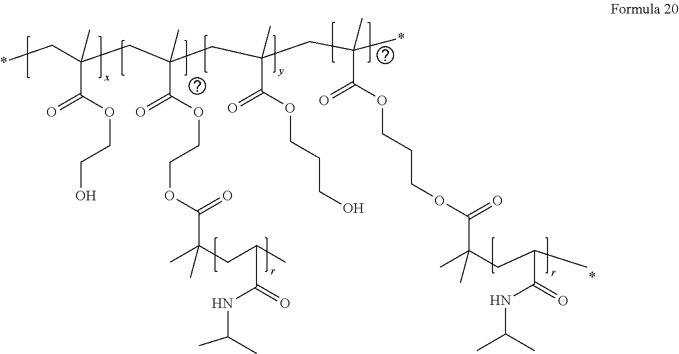

### Example 9: Synthesis of Tri-Arm Poly(HEMA₉₀-co-HPMA₁₀)

In a 250 mL three-neck round bottom flask, 0.3 g (0.56 mmol) of initiator prepared in Example 1 was mixed with 2-hydroxyethyl methacrylate 19.6 g (151 mmol) and 2.41 g (16.8 mmol) of hydroxypropyl methacrylate in 23 mL of methanol. The solution was degassed with nitrogen for 60 min, then 0.24 g (1.68 mmol) of CuBr and 0.52 g (3.36 mmol) of 2,2′-bipyridine were added under nitrogen flow to start polymerisation. After about 20 hr, the reaction was stopped by exposure to the air and diluted with methanol. The mixture was precipitated into deionised water to remove copper catalyst and solid precipitation was received. The solid was dissolved in MeOH and precipitated into water. The extraction was repeated three times. The received polymer was vacuum dried at 40° C. overnight and 18.8 g of solid was received.

### Example 10: Synthesis of Poly (I-(HEMA₉₀-co-HPMA₁₀)₃) Macroinitiator

In a 250 mL round bottom flask, 10 g (0.25 mmol) of tri-arm poly(I-(HEMA-HPMA)₃) obtained from Example 8 was dissolved into 50 mL of anhydrous N-methyl pyrrolidinone (NMP) under magnetic stirring, followed by adding 1.15 mL of triethylamine. The flask was placed in an ice-water bath, then 0.93 mL of α-bromoisobutyryl bromide to target 10 unit of bromide per arm was added dropwise through a syringe over about 10 min. The reaction solution became turbid quickly and was stirred at room temperature overnight. The reaction mixture was precipitated dropwise into deionised water 500 mL to remove NMP and triethylamine salt. The collected solid was dissolved in 30 mL of NMP and the precipitation was repeated again. The solid was collected and vacuum dried at 40° C. for two days.

### Example 11: Synthesis of Comb-Like Tri-Arm Poly(I-(HEMA₉₀-co-HPMA₁₀)-Graft-(NIPAAM₂₀)₁₀)₃ by ATRP

In a 100 mL of three-neck round bottom flask, 3.0 g of poly(HEMA-HPMA) macroinitiator prepared in Example 9 and 5.1 g of NIPAAM monomer were dissolved in 50 mL of NMP. The mixture was degassed with nitrogen for 60 min, then CuBr 0.32 g and PMDETA 0.39 g were added to start the polymerisation. The reaction was left running overnight. The mixture was then dialysed against deionised water for four to five days to remove unreacted monomer and catalysts. The solution was freeze dried and polymer solid was received (Formula 17) (see also scheme 5).

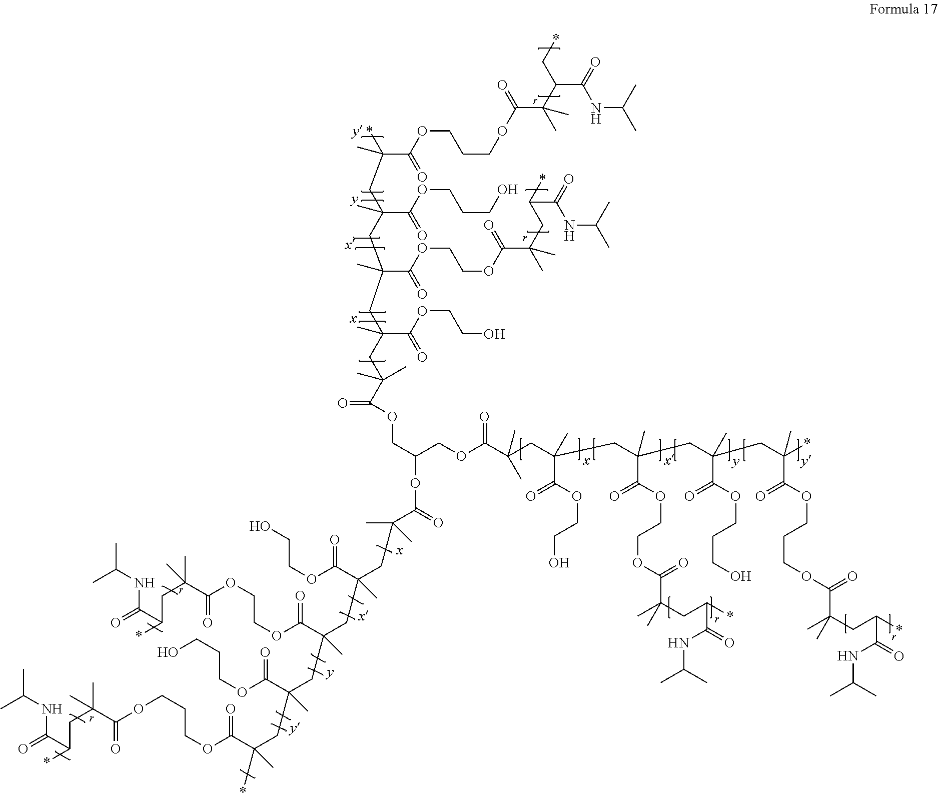

### Example 12: Sample Preparation and In Vitro Testing

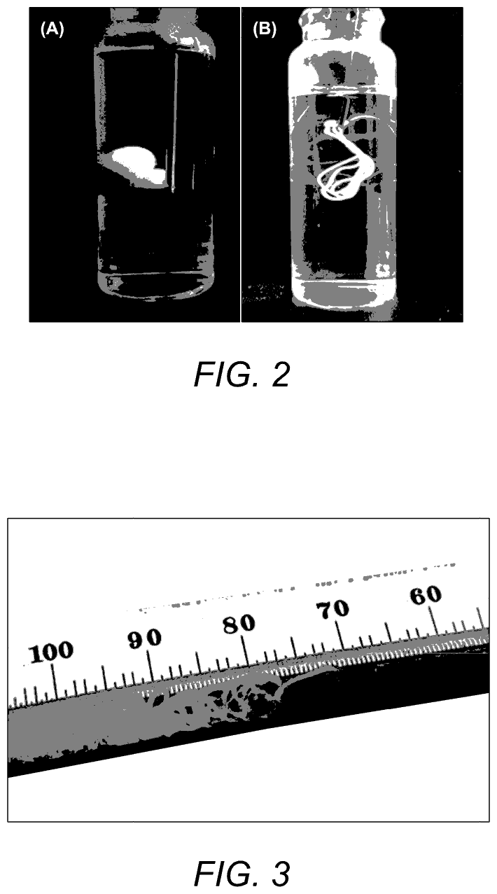

Samples of polymer solid generated as described above were weighed into a 30 mL vial, deionised water was added to provide a 10% w/w polymer solution. The vial was placed in a cold water bath and magnetically stirred until a uniform solution was obtained. The solutions were translucent and of low viscosity. The gelation capability of the polymer solution was tested by injecting the solution into a phosphate buffered saline medium at 37° C. through an 18G needle. As shown in FIG. 1, solutions instantly became a white hydrogel. The gel formed from polymers with lower hydrophobicity (such as those lacking HPMA) tended to form a soft lump shown in FIG. 2A, whilst the more hydrophobic polymers (in this case with 10% HPMA mole/mole in the PA block) tended to be harder and string like, shown in FIG. 2). A press test with the needle indicated that the gel strength was strong enough not to be easily broken. Table 1 records the observed properties of example polymers delivered into PBS solutions at 37° C. Polymer solutions were prepared in either Omnipaque™ 300 aqueous contrast medium, used at full strength, or in deionized water.

TABLE 1

| | TEST RESULTS |
|---|---|
| LINEAR POLYMERS | |
| I-(HEMA₅₀-b-NIPAAM₂₀₀)₂ | Soft gel string when delivered with contrast, |
| | solidified and volume reduced. |
| I-(HEMA₉₀-co-HPMA₁₀)-g-(NIPAAM₂₀)₁₀ | Medium strength no fragments |
| I-(HEMA₈₀-co-HPMA₂₀)-g-(NIPAAM₂₀)₁₀ | Medium strength no fragments |
| I-((HEMA₄₅-co-HPMA₅)-NIPAAM₁₀₀)₂ | Rigid gel string in 37° C. PBS, solidified quickly and no |
| | volume reduction. |
| I-((HEMA₄₅-co-HPMA₅)-NIPAAM₂₀₀)₂ | More rigid gel string when delivered with contrast, |
| | solidified quickly and no significant volume reduction |
| | observed over 3 days (gel kept in PBS 37° C.) |
| I-((HEMA_{44.5}-co-HPMA_{0.5})-NIPAAM₂₀₀)₂ | Relatively soft gel string in 37° C. PBS, solidified quickly |
| | and volume reduction observed. |
| STAR POLYMERS | |
| I-[HEMA₁₀₀-g-(NIPAAM₂₀)₁₀]₃ | Solid gel string delivered with contrast. High gel strength |
| | without fragments observed. Slight gel volume reduction |
| | after delivery. |
| I-[HEMA₁₀₀-g-(NIPAAM₃₀)₁₀]₃ | Medium to high gel strength, some fragments observed |
| | during delivery. |
| I-[HEMA₁₀₀-g-(NIPAAM₁₀)₃₀]₃ | Weak gel when delivered into PBS, fragments generated. |
| I-[HEMA₁₀₀-g-(NIPAAM₂₀)₃₀]₃ | Weak to medium gel strength, fragments observed. |
| I-[HEMA₁₀₀-b-NIPAAM₁₀₀]₃ | Relatively soft gel, medium strength, volume reduction |
| | observed. |
| I-[HEMA₁₀₀-b-NIPAAM₂₀₀]₃ | Soft gel, medium gel strength, volume reduction |
| | observed. |
| I-[(HEMA₉₀-b-HPMA₁₀)-g-(NIPAAM₂₀)₁₀]₃ | Strong gel no fragmentation. |
| I-[(HEMA₈₀-b-HPMA₂₀)-g-(NIPAAM₂₀)₁₀]₃ | Strong gel no fragmentation, but slower to dissolve. |

### Example 13: Rheology

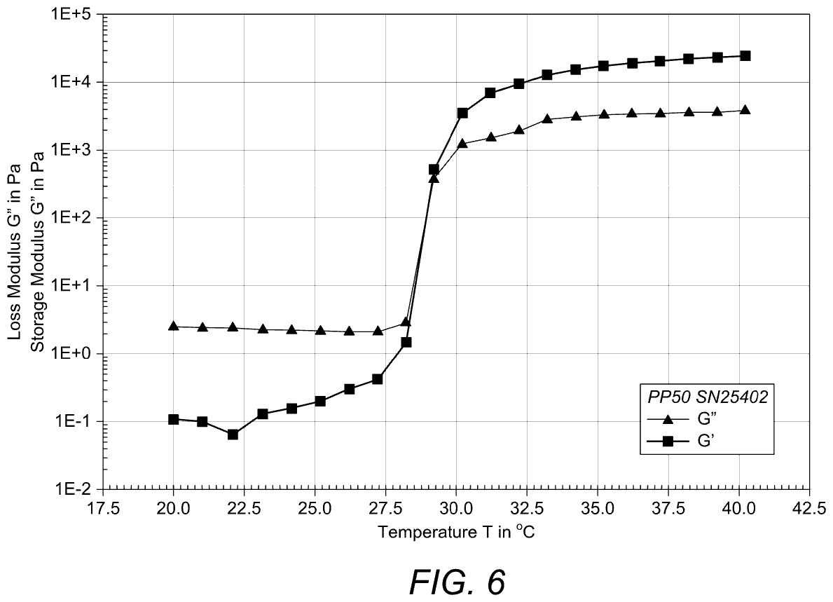

A sample of I-((HEMA_{44.5}-co-HPMA_{0.5})-NIPAAM₂₀₀)₂ aqueous solution (15% w/w) was tested by rheometer using the oscillation method, and the temperature ramping result was shown in FIG. 6. It shows that the sol-gel transition temperature is around 29° C. and the storage modulus of the gel is about 20,000 Pa and loss modulus is 4,000 Pa at 37° C., which suggests that a solid gel could be formed when the sample was placed at body temperature.

### Example 14: Delivery of Polymer in a Flow Model

To prepare a polymer solution with contrast medium, polymer solid was weighed into a 30 mL vial and contrast medium—Omnipaque 300 (647 mg/ml iohexol) was added to make the polymer concentration 10% w/w. The vial was placed in a cold water bath and magnetically stirred to dissolve the polymer. The obtained polymer solution was transferred into a syringe for delivery test with 2.4 Fr catheter.

In a flow model, a 5 mm diameter silicon tubing was immersed in a water bath at 37° C. A sponge was placed in the middle of the tubing, to mimic a collection of small vessels such as the rete mirabile. PBS medium was pumped through the sponge tubing. A bypass enabled the flow rate through the sponge to be controlled at 150 m/min, and actual flow rate was monitored by a flow meter. The open end of a 2.4 Fr catheter was placed in front of the sponge and the polymer solution was injected into the tubing. The polymer solution transitioned into a hydrogel thread as it exited the tip of the catheter in the flowing medium, building up and forming a plug, occluding the flow path and causing the flowmeter reading to drop to 0 within 1-2 min (FIG. 3).

### Example 15: Formulation of Dried Polymer

Samples of polymer powder were prepared by either grinding lyophilised solid or spray drying from polymer solutions to provide a particle size distribution of between 10-600 microns. The powder (0.05 g) with or without glycerol (0.025 g) were weighed into a 1 mL syringe. In a second 1 mL syringe, 0.925 g of contrast solution was weighed. The two syringes were linked together through a 3-way stopcock and air was carefully removed. The contrast solution was quickly mixed with polymer solid by passing the composition back and forth between the two syringes. The syringe was placed in a fridge at 2-8° C. for 5 min to cool the contents and allow better dissolution. The solution was further mixed for around 20 times. A catheter was linked to the syringe to deliver the mixture to PBS, 37° C., and string-like hydrogels were formed in the PBS.

### Example 16 Preparation and Properties of Oily Emulsions

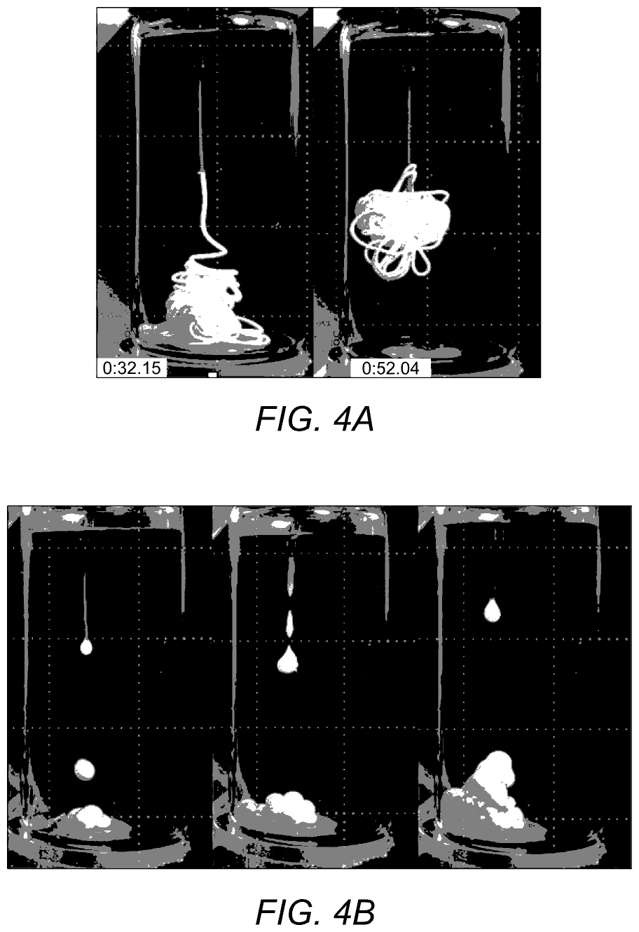

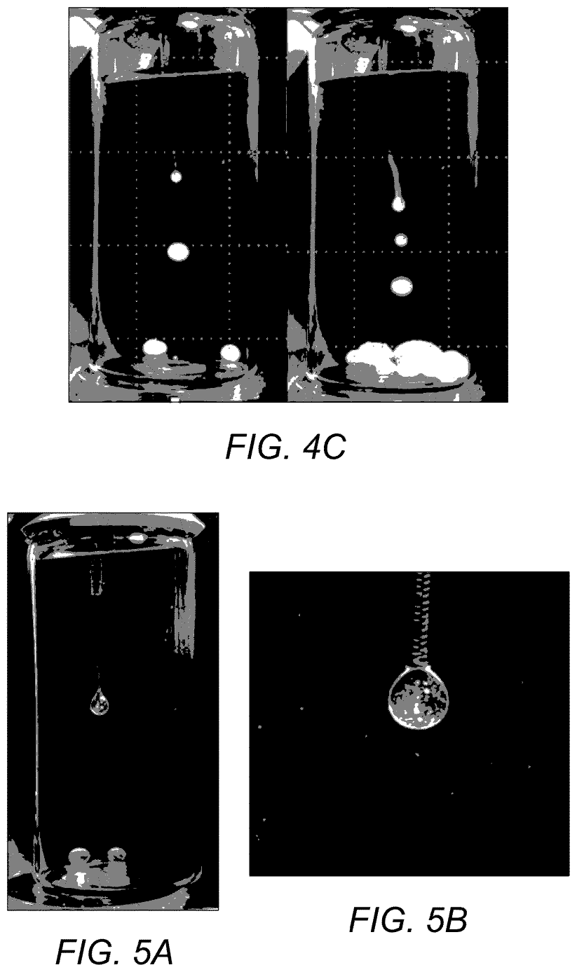

A 10% aqueous solution of I-((HEMA₄₅-co-HPMA₅)-NIPAAM₂₀₀)₂ prepared and mixed with iodised poppy seed oil (Lipiodol® Guerbet, Paris, France) at various ratios (see Table 3) by repeatedly passing the mixture back and forth between two syringes through a 3-way stopcock, to obtain a uniform emulsion. The emulsion was slowly injected into PBS buffer at 37° C., and the characteristics of the product were observed. FIGS. 4A to 4C illustrate the products formed.

### Example 17 Catheter Delivery of Emulsion Based on Linear Polymers in a Flow Model

Samples of the emulsions prepared according to Example 16 were delivered through a 2.7 Fr catheter in a flow model according to Example 14, mimicking vascular embolisation in PBS at 37° C. Observations are recorded in Table 3.

TABLE 3

| Mixture ratio | | |
|---|---|---|
| (polymer:oil) | Observations upon injection into | Observations upon |
| (v/v) | PBS at 37° C. | injection into flow model |
| 1:0.5 | String like hydrogel forms | Moderate force required to |
| | immediately (FIG. 1) | deliver emulsion. No |
| | | Lipiodol ® escape observed |
| 1:1 | Blob-like fluid gel forms | Less force required for |
| | immediately (FIG. 2). Some | delivery than 1:0.5 ratio but |
| | Lipiodol ® appears to escape from | Lipiodol ® escape observed |
| | the hydrogel. | |
| 1:2 | Blob-like fluid gel forms | Less force required for |
| | immediately (FIG. 3). Some | delivery than 1:0.5 ratio but |
| | Lipiodol ® appears to escape from | Lipiodol ® escape observed |
| | the hydrogel. | |

### Example 16. Catheter Delivery of Lipiodol® Oil with Dry Form of Polymer Particles into PBS

Samples of the Lipiodol® oil with dry polymer powder of I-((HEMA_{49.5}-co-HPMA_{0.5})-NIPAAM₂₀₀)₂ prepared by mixing directly Lipiodol® oil and 200-500 μm polymer particles between two syringes as described above. The formulation was delivered through a 2.7 Fr catheter into PBS buffer at 37° C. (FIG. 5). It is observed that the polymer powder was suspended inside the oil droplets.

### Example 17. Release of Drug Mimic from the Hydrogel Oily Emulsions

The water-soluble dye Safronin O was dissolved in a 10% aqueous solution of the copolymer I-((HEMA_{49.5}-co-HPMA_{0.5})-NIPAAM₂₀₀)₂. The solution was then mixed with Lipiodol® in a ratio of 1:1 (v/v) to form a water-in-oil emulsion which had a pinkish colour. The emulsion was injected into PBS buffer at 37° C., and a hydrogel fluid formed immediately. It was observed that the red dye gradually released from the injected hydrogel into PBS, and the dye was completely released after 48 hr.

Similarly an oil soluble dye Sudan IV was dissolved in Lipiodol®, followed by mixing with a 10% aqueous solution of I-((HEMA_{49.5}-co-HPMA_{0.5})-NIPAAM₂₀₀)₂ in 1:1 ratio to obtain a pink coloured water-in-oil emulsion. The emulsion was injected into PBS buffer at 37° C., and a hydrogel fluid formed immediately. No red dye was released from the injected hydrogel in PBS, and there was no obvious dye release into PBS even after 48 hr.

### Example 18: Synthesis of Poly(NIPAAM-[GMA-St-BMA]-NIPAAM) and Poly(NIPMAM-[GMA-St-BMA]-NIPMAM) Copolymers

A. Synthesis of poly(GMA₉₅-st-BMA₅)

0.1 g (0.3 mmol) of initiator diethyl meso-2,5-dibromoadipate was mixed with glycerol monomethacrylate 4.7 g (29.3 mmol) and butyl methacrylate 0.2 g (1.4 mmol) in a 50 mL of three-neck round bottom flask, followed by adding 5 mL of methanol. After the mixture was degassed with nitrogen gas under magnetic stirring for 60 min, 0.08 g (0.6 mmol) of Cu(I)Br and 0.1 g (0.6 mmol) of N,N,N′,N″,N″-pentamethyldiethylenetriamine were added into the flask to start the polymerisation. It is observed that the temperature of the reaction mixture increased initially accompanied by an increasing solution viscosity.

After about 20 hr, the reaction was stopped by exposure to the air dilution with methanol. The mixture was passed through a silica gel column to remove the copper catalyst, and the methanol was removed by rotary evaporation. After further vacuum drying overnight at 40° C., 4.1 g solid was received.

B. Synthesis of Poly(NIPAAM₂₀₀-[GMA₉₅-st-BMA₅]-NIPAAM₂₀₀) Copolymer.

In a 50 mL three-neck round bottom flask, 1.9 g of poly(GMA-st-BMA) macroinitiator obtained from the reaction above was dissolved into 9 mL of methanol under magnetic stirring. Then 5.3 g of N-isopropylacrylamide monomer was added into the flask and dissolved. After the mixture was degassed with nitrogen for 60 min, the flask was placed in an ice-water bath, and 0.03 g (0.23 mmol) of Cu(I)Br and 0.06 g (0.23 mmol) of 1,4,8,11-Tetramethyl-1,4,8,11-tetraazacyclotetradecane were added to start polymerisation. The reaction solution became viscous quickly and was stirred overnight under nitrogen atmosphere.

After about 20 hr the reaction was stopped by exposure to the air and the addition of 100 mL of methanol to dilute the viscous solution. The polymer was purified by ultrafiltration against water and the obtained solution was freeze-dried over 48 hr. Approximately 5.5 g of solid was collected.

C. Synthesis of poly(NIPMAM₂₀₀-[GMA₉₅-st-BMA₅]-NIPMAM₂₀₀) copolymer

The product was prepared by the reaction of the poly(GMA-st-BMA) macroinitiator prepared above, with N-isopropylmethacrylamide monomer in the presence of a catalyst Cu(I)Br and ligand 1,4,8,11-Tetramethyl-1,4,8,11-tetraazacyclotetradecane in a similar manner to the above preparation. A yellowish polymer solid was obtained after freeze drying.

Both of the polymers were soluble in cold water and showed phase changes of polymer aggregation and gel formation at 37° C.

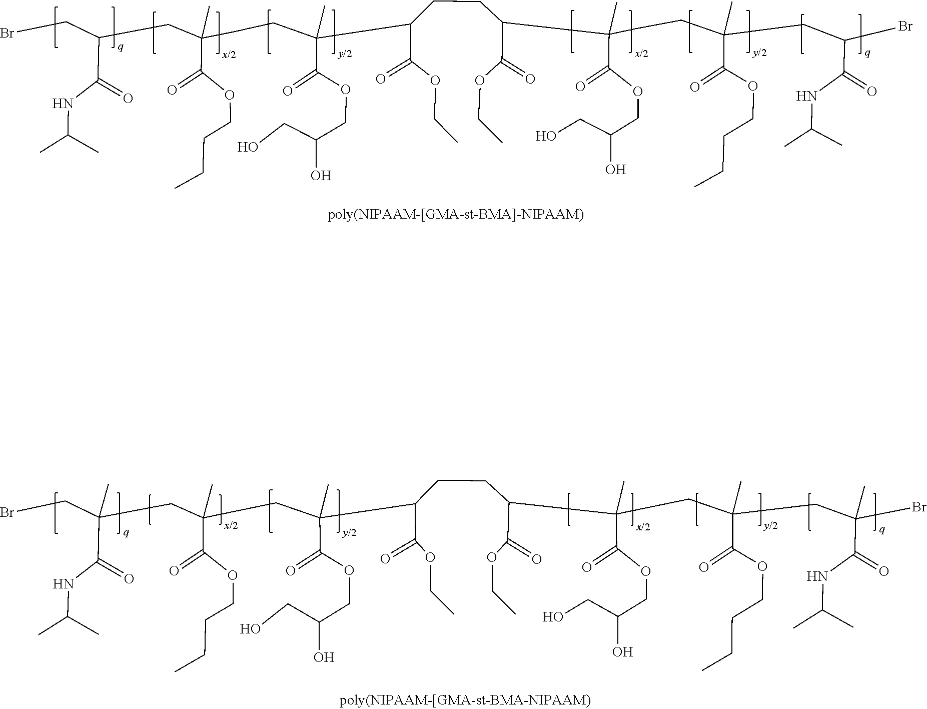

1. A block co-polymer comprising a First Block comprising A monomer and optionally P monomer and a Second Block consisting of N monomer; wherein P monomer is more hydrophobic than A monomer; wherein the second block is a thermally responsive block; wherein
A monomer is selected from acrylates, methacrylates, acrylamides and methacrylamides; preferably A monomer is selected from the group consisting of an acrylate ester, a methacrylate ester, an N-substituted acrylamide and an N-substituted methacrylamide;
P monomer is selected from the group consisting of acrylates, methacrylates, acrylamides and methacrylamides; preferably P monomer is selected from the group consisting of an acrylate ester, a methacrylate ester, an N-substituted acrylamide and an N-substituted methacrylamide;
wherein P is more hydrophobic than A; and wherein;
Second Bock is selected from poly(N-isopropylacrylamide), (pNIPAAM), poly(N-isopropylmethacrylamide) (pNPMAM), poly(N,N-diethyl acrylamide) (pDEA), poly(2-(dimethylamino)ethyl methacrylate) (pDMAEMA), poly(PEG methacrylate) (pPEGMA), poly(N-vinylcaprolactam), poly(2-isopropyl-2-oxazoline), poly(vinyl methyl ether), poly(l-lactic acid)-poly(ethylene glycol)-poly(1-lactic acid) (PLLA-PEG-PLLA), hydroxypropylcellulose and poly(ethyleneoxide)-poly(propyleneoxide)-poly (ethyleneoxide); preferably poly(N-isopropylacrylamide) or poly(N,N-diethyl acrylamide).

2. The block co-polymer according to claim 1, wherein the A monomer is selected from the group consisting of 2-hydroxyethyl methacrylate (HEMA), N-(2-hydroxyethyl) acrylamide (HEA), 1,3-dihydroxyproyl methacrylate (1,3DHPMA) 2,3-dihydroxy propylmethacrylate (2,3DHPMA), glycerol monomethacrylate (GMA) and N-(2-hydroxyethyl) methacrylamide (HEMAm); particularly 1,3DHPMA, 2,3DHPMA, GMA and HEMA; particularly HEMA; the P monomer is selected from 3-hydroxypropyl methacrylate, 2-hydroxyisopropyl methacrylate, 1-hydroxy-2-propanyl methacrylate and 2-hydroxypropyl methacrylate, or a mixture of any two or more of the forgoing isomers (HPMA); N-(2-hydroxypropyl) methacrylamide (HPMAm), N-(3-hydroxy propyl)acrylamide (HPA) and butyl methacrylate (BMA); and the N monomer is selected from N-NIPAAM, NIPMAM and DEA; particularly the A monomer is HEMA, the P monomer is HPMA and the Second Block is poly NIPAAM.

3. The block co-polymer according to claim 1 or 2 wherein the number of A monomers in a First Block is 20 to 600; preferably 30 to 200; more preferably 60 to 120; or 70 to 100; and, the number of P monomers in a First Block is either 0 or is between 0 and 500; preferably 1 or 2 to 100 more preferably 3-30; more preferably >0 and less than or equal to 10.

4. The block co-polymer according to any preceding claim, wherein the number of N monomers present as second block is 1 to 800; preferably 3 to 600 more preferably 5 to 400.

5. The block co-polymer according to any of claims 1 to 3, wherein Second Block is grafted to First Block and the total number of N monomers grafted to a First Block is 3 to 500, preferably 5 to 200, and more preferably 5 to 50.

6. The block co-polymer according to any of claims 1 to 3, wherein Second Block is present as an extension to First Block and the number of N monomers in the Second Block is 1 to 800; preferably 20 to 600; more preferably 50 to 400.

7. The block co-polymer according to any preceding claim, wherein the First Block comprises A monomer and P monomer and the ratio of total A:total P:total N is 30-500:(0 or 1)-200:100-600 on a molar basis.

8. The block co-polymer according to any preceding claim, wherein the First Block comprises A monomer and P monomer and wherein A is 70 to 99.9% of the First Block on a molar basis.

9. The block co-polymer according to any preceding claim, wherein the P monomer has an X Log P3 value of at least 0.10 higher than the A monomer.

10. The block co-polymer according to any preceding claim, wherein the ratio of First Block monomers to Second Block monomers is 1:0.1 to 1:8 mol/mol.

11. The block co-polymer according to any preceding claim, in which Second Block is pendant from First Block.

12. The block co-polymer according to any of claims 1 to 10, in which Second Block is an extension of First Block.

13. The block co-polymer according to any preceding claim, wherein First Block is an (AP) block comprising both A and P and is an (A-b-P) block, (P-b-A) block or (A-co-P) block, and the co-polymer is (AP)-b-N or (AP)-g-N.

14. A block co-polymer according to any preceding claim, which is either in a linear format, comprising a single First Block or two identical First Blocks covalently coupled in a linear reflected arrangement about a central hub; or a star format comprising m identical First Blocks individually covalently coupled to a central hub, wherein m is an integer from 3-10.

15. A block co-polymer of the formula I: 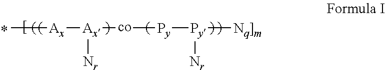
wherein:
* represents the site of attachment to the residue of a polymerisation initiator;
A is selected from the group consisting of acrylates, methacrylates, acrylamides, methacrylamides;
P is selected from the group consisting of acrylates, methacrylates, acrylamides, methacrylamides;
wherein P is more hydrophobic than A as measured by X Log P3;
N is a monomer forming a thermally responsive block and N_{q} and Nᵣ are terminal, thermally responsive blocks selected from the group consisting of poly(N-isopropylacrylamide), (NIPAAM), poly(N-isopropylmethacrylamide) (NIPMAM), poly(N,N-diethyl acrylamide) (DEA), poly(2-(dimethylamino)ethyl methacrylate) (pDMAEMA), poly(PEG methacrylate) (PEGMA), poly(N-vinylcaprolactam), poly(2-isopropyl-2-oxazoline), poly(vinyl methyl ether), poly(l-lactic acid)-poly(ethylene glycol)-poly(1-lactic acid) (PLLA-PEG-PLLA), hydroxypropylcellulose and poly(ethyleneoxide)-poly(propyleneoxide)-poly (ethyleneoxide); preferably poly(N-isopropylacrylamide) or poly(N,N-diethyl acrylamide);
wherein:
the sum of x and x′ is the number of A monomers in a block, and is an integer from 20 to 600, preferably 30-200, yet more preferably 60 to 120; or 70 to 100;
The sum of y and y′ is the number of P monomers in a block; y and y′ are both 0 when P is absent; when P is present (y+y′) is either 0 or is an integer from 1 to 500, preferably 2 to 100 more preferably 3-30;
The sum of x′ and y′ is the total number of grafts in the AP Block and is either 0 or is an integer from 2 to 500, preferably 6 to 275, more preferably 8 to 130;
q is the number of N monomers in an extension block, and is 0 or is an integer from 1-800, preferably 20-600, more preferably, 50-400;
r is the number of N monomers in a pendant block and is 0, or is an integer from 3 to 500, preferably 5 to 200, and more preferably 5 to 50; and q and r may not both be zero at the same time;
if r>0 then q is an integer not greater than 2*r, preferably not greater than r;
m is an integer from 1 to 10, preferably from 1 to 6; more preferably from 1 to 4; and yet more preferably is 1, 2 or 3; and wherein
parentheses represent an integral block with hydrophobic/hydrophilic function formed in a sequence of synthesis; and square brackets enclose an arm of branched structures; and wherein the order of A and P may be reversed when A-P is in the form of blocks.

15. The block co-polymer according to claim 14, wherein A is selected from the group consisting of 2-hydroxyethyl methacrylate (HEMA), N-(2-hydroxyethyl) acrylamide (HEA), 1,3-dihydroxyproyl methacrylate (1,3DHPMA) 2,3-dihydroxy propylmethacrylate (2,3DHPMA), glycerol monomethacrylate (GMA) and N-(2-hydroxyethyl) methacrylamide (HEMAm); preferably HEMA, 1,3DHPMA, 2,3DHPMA and GMA; preferably HEMA

16. The block co-polymer according to claim 14 or 15, wherein P is selected from the group consisting of 3-hydroxypropyl methacrylate, 2-hydroxyisopropyl methacrylate, 1-hydroxy-2-propanyl methacrylate and 2-hydroxypropyl methacrylate, or a mixture of any two or more of the forgoing isomers (HPMA); N-(2-hydroxypropyl) methacrylamide (HPMAm), butyl methacrylate (BMA)and N-(3-hydroxy propyl)acrylamide (HPA).

17. The block co-polymer according to claims 14 to 16, wherein the N-block is selected from the group consisting of poly(NIPAAM), poly NIPMAM, and poly(DEA); preferably poly(NIPAAM) and poly NIPMAM.

18. The block co-polymer according to claims 14 to 17, wherein A is HEMA, P is HPMA and N is NIPAAM or A is selected from 1,3DHPMA, 2,3DHPMA and GMA; P is BMA and N is NIPAAM or A is selected from 1,3DHPMA, 2,3DHPMA and GMA, P is BMA and N is NIPMAM

19. A block co-polymer according to any of claims 14 to 18, in which (y+y′)>0 and the (AP) block is an (A-b-P) block, (P-b-A) block or (A-co-P) block and the co-polymer is (AP)-b-N or (AP)-g-N.

20. A block co-polymer selected from: 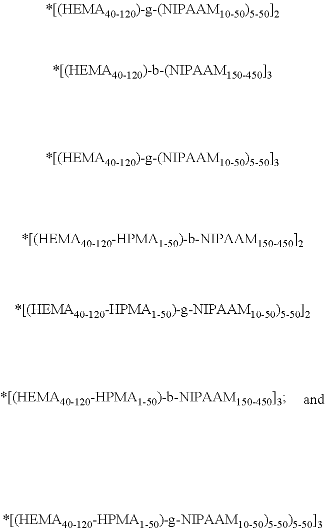
wherein values for the number of HEMA, HPMA and NIPAAM monomers are integers

21. A block co-polymer selected from: 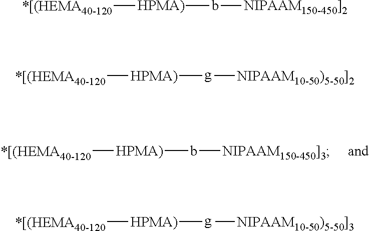
wherein values for the number of HEMA, HPMA and NIPAAM monomers are ranges including all fractional values there between; and wherein the number of HPMA monomers in the AP block is >0 and <=50

22. A block co-polymer selected from:

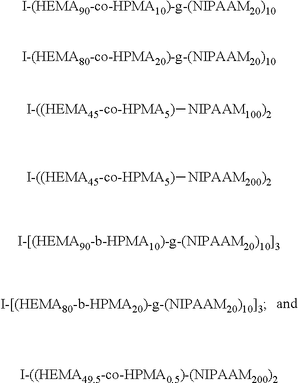

23. A process for the preparation of a block copolymer comprising polymerizing A monomer and optionally P monomer to provide First Blocks which are either A blocks or AP blocks wherein polymerization is initiated with a polymerization initiator, I, having m sites of polymerization initiation;
and reacting N monomers with First Block to provide a block co-polymer comprising First, A blocks or AP blocks and Second, N blocks; wherein:
A monomer is reacted at an I:A ratio of 1:(20-600)m preferably 1:(30 to 200)m more preferably 1:(60 to 120)m and yet more preferably at 1:(70 to 100)m mol/mol;
P monomer, where present, is reacted at an I:P ratio of 1:(0-500)m, preferably 1:(2 to 100)m, more preferably 1:(3-30)m mol/mol; and
N monomer is reacted at an I:N ratio of 1:(1 to 800)m, preferably 1:(3 to 600)m, more preferably 1:(5 to 400)m mol/mol; and
m is an integer from 1 to 10; and
wherein
A monomer is selected from acrylates, methacrylates, acrylamides and methacrylamides; preferably A monomer is selected from the group consisting of an acrylate ester, a methacrylate ester, an N-substituted acrylamide and an N-substituted methacrylamide;
P monomer is selected from the group consisting of acrylates, methacrylates, acrylamides and methacrylamides; preferably P monomer is selected from the group consisting of an acrylate ester, a methacrylate ester, an N-substituted acrylamide and an N-substituted methacrylamide, wherein P is more hydrophobic than A; and wherein
N monomer is selected from NIPAAM, NIPMAM, N,N-diethyl acrylamide, 2-(dimethylamino)ethyl methacrylate—(DMAEMA), N-vinyl caprolactam, 2-isopropyl-2-oxazoline, vinyl methyl ether, 1-lactic acid-poly(ethylene glycol)-poly(1-lactic acid) (PLLA-PEG-PLLA), N-vinylcaprolactam and 2-isopropyl-2-oxazoline, vinyl methyl ether; preferably (NIPAAM), NIPMAM or N,N-diethyl acrylamide (DEA); particularly NIPAAM.

24. A process for the preparation of a block co-polymer comprising polymerising A monomer or A and P monomers to form a first block comprising A monomer and, optionally, P monomer, of the formula 10 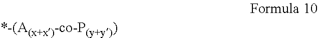
and either
(a) extending an Nq block from the (A_{(x+x′)}-co-P_{(y+y′})) block to form a polymer of the general formula 11 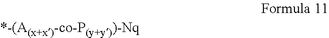
or;
(b) extending at least one Nᵣ graft from the AP block to form a co-polymer comprising at least one Nᵣ graft; 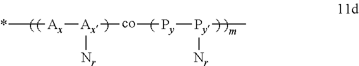
wherein * represents the residue of an optional initiator molecule, configured to support initiation of polymerisation from m functional groups; and wherein m is an integer from 1 to 10.

25. The process according to claim 23 or 24, wherein the polymerisation is carried out by a RAFT, ATRP or conventional free radical polymerisation; preferably carried out by ATRP.

26. A block co-polymer prepared according to any of claims 23 to 25.

27. A bulk polymer composition comprising a block co-polymer according to any of claims 1 to 22 or 26.

28. A dried composition comprising a block co-polymer according to any of claims 1-22 or 26 or bulk polymer composition according to claim 27.

29. A dried composition according to claim 28, comprising less than 0.1% w/w water or other solvent.

30. An aqueous composition comprising a block co-polymer according to any of claims 1-22 or 26 or bulk polymer composition according to claim 27.

31. The aqueous composition according to claim 30, wherein the co-polymer or bulk polymer composition is in the sol form at 25° C. and in the gel form at 37° C.

32. The composition according to any of claims 27 to 31, which additionally comprises at least one imaging agent, at least one therapeutic or diagnostic radioisotope and/or at least one chemotherapeutic agent.

33. A composition comprising a block co-polymer according to any of claims 1-22 or 26 or bulk polymer composition according to claim 27 and an iodinated contrast agent wherein the ratio of contrast agent to co-polymer or polymer composition is between 0.1 to 110 on a weight for weight basis, preferably 1 to 10, more preferably 1.5 to 5.

34. A composition comprising a block co-polymer according to any of claims 1-20 or 24 or a bulk polymer composition according to claim 25 and an ethiodized poppy seed oil.

35. A composition according to claim 34, in the form of an oil in water emulsion.

36. A composition according to claim 34, wherein the block co-polymer or a bulk polymer composition is in particulate form.

37. An embolic agent comprising a block co-polymer according to any of claims 1-22 or 26 or bulk polymer composition according to claim 27, or a composition according to any of claims 27 to 36.
